(19) United States
(12) Patent Application Publication
(10) Pub. No.: US 2009/0180959 A1
(43) Pub. Date: Jul. 16, 2009

(54) VDCC Gamma-8 Ion Channel

(76) Inventors: John Dixon, Cambridge (GB); Justin Powell, Cambridge (GB); Malcolm Sheardown, Cambridge (GB); Nicola Brice, Cambridge (GB); Jennifer Marie Horwood, Cambridge (GB); Isabelle Malinge, Cambridge (GB); Haiping Liu, Singapore (SG); Kok Siu Foo, Singapore (SG)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/996,983

(22) PCT Filed: Sep. 4, 2006
(86) PCT No.: PCT/GB06/03263

§ 371 Date: Aug. 15, 2008

(30) Foreign Application Priority Data
Sep. 2, 2005 (GB) 0517916.3

(51) Int. Cl. A61K 49/00; G01N 33/68; C12Q 1/02; A01K 67/027; C12Q 1/68

(52) U.S. Cl. 424/9.1; 436/86; 435/29; 800/13; 800/9; 435/6

(57) ABSTRACT

VDCC γ-8 polypeptides comprising the amino acid sequence shown in SEQ ID NO. 3, SEQ ID NO. 5 or SEQ ID NO: 7, and homologues, variants and derivatives thereof are provided. Nucleic acids capable of encoding VDCC γ-8 polypeptide are also disclosed, in particular, those comprising the nucleic acid sequences shown in SEQ ID No. 1, SEQ ID No. 2, SEQ ID NO. 4 or SEQ ID NO: 6. These polypeptides and polynucleotides are implicated in mental illness and are useful in screening for therapeutic agents useful in treating mental illness.

### FIELD OF THE INVENTION

The invention relates to ion channel nucleic acids and polypeptides. The ion channel is hereinafter referred to as “voltage-dependant calcium channel gamma-8”, “VDCC γ-8” or “γ-8”. The invention also relates to inhibiting or activating the action of these nucleic acids and polypeptides, in particular to provide candidates for the therapy of mental illness.

### BACKGROUND OF THE INVENTION

Calcium channels are crucial in the regulation of a wide range of cellular functions, including but not exclusively, muscle contraction, neurotransmitter release, hormone secretion, gene expression, progression through the cell cycle and cell death. Intracellular calcium is very low at rest, and changes in this to produce a transient rise in intracellular calcium can act as a second messenger, to activate a diverse range of cellular processes. Either voltage- or ligand-gated calcium channels can mediate this increase in intracellular calcium. Voltage-dependent calcium channels can be split into two broad groups: low (or T-type) and high threshold-activated channels. High threshold channels are subdivided into L, N, P, Q, and R channels, depending on their subunit composition.

Voltage dependent calcium channels are heteromeric channels composed of a pore forming alpha-1 subunit and the accessory subunits beta and alpha-2-delta. In some situations, a gamma subunit is also present (Caterall, Curr. Opin Neurobiol. 1991: 1; 5-13). Gamma subunit proteins are integral membrane proteins that are thought to stabilize the calcium channel in an inactive (closed) state. There are several gamma subunit proteins and this VDCC-γ8 gene is a member of the neuronal calcium channel gamma subunit gene subfamily of the PMP-22/EMP/MP20 family, located in a cluster with two similar gamma subunit-encoding genes. It is believed that non-AUG translation initiation can occur for the VDCC-γ8 transcript. The VDCC-γ8 subunit contains a postsynaptic density-95, discs large, zonula occludens (PDZ)-binding motif. VDCC-γ8 appears later in development and progressively increases during animal maturation (and is not seen in embryonic development cerebral cortex, see Tomita et al., J Cell Biol, 2003: 161; 805-816), as does stargazin and VDCC-γ3. This is in context to VDCC-γ4 which is expressed embryonically and gradually declines during animal maturation.

γ-8, along with Stargazin, γ-3 and γ-4 belong to a family of transmembrane AMPA regulatory proteins (TARPS) that mediate surface expression of AMPA receptors (˜75% decrease in surface expression of GluRs in cerebellar cells from stargazer mice, whereas total GluR levels are only reduced by 10-20%.

Mental illness is a term used to define brain disorders, in particular those involving affective or emotional instability, behavioural dysregulation and/or cognitive impairment. Mental illness can be debilitating to the sufferer and caring for one suffering a significant emotional and financial burden.

Psychosis is a term defining a mental state having severely impaired thought and perception. A psychotic episode is debilitating and commonly involves hallucinations, paranoia, delusions, disorganised thinking and impairment of social interaction. Psychoses generally involve a loss of contact with reality, which can be highly destructive to the sufferer. Psychoses manifest in conditions including bipolar disorder, severe clinical depression and schizophrenia. These conditions are not well understood and effective therapies to prevent and treat them are required. In particular, there is a significant unmet medical need in the therapy of the negative symptoms schizophrenia. The most commonly used drugs, clozapine and olanzapine, take 2-4 weeks to be fully efficacious and have relatively weak effects on the negative symptoms of schizophrenia.

Epilepsy is a mental illness, often also referred to as a developmental disability, characterised by recurrent epileptic seizures. This chronic neurological condition affects many millions of people worldwide and, although drugs control the symptoms exist, there is no cure.

There remains a strong need for effective therapies to prevent and treat mental illness, in particular anti-psychotic and anti-epileptic therapies.

### SUMMARY OF THE INVENTION

The present invention is based on the surprising discovery that VDCC γ-8 is implicated in mental illness and is therefore a useful therapeutic target. In particular, VDCC γ-8 is implicated in diseases involving psychosis such as schizophrenia, and epilepsy.

According to a first aspect of the present invention, a method of identifying an agent suitable for therapy of a mental illness, comprises the step of determining whether a candidate agent affects the activity of voltage-dependant calcium-channel gamma-8.

### BRIEF DESCRIPTION OF THE DRAWINGS

FIG. 1 is a diagram showing the knockout vector.

FIG. 2 is a graph showing the results of the rotarod test for knockout mice (mutant, −/−) compared with wild-type mice (wt, +/+).

FIG. 3A is a graph showing the results a 1-hr session in LABORAS® (Laboratory Animal Behaviour Observation Registration and Analysis System) for knockout mice (mutant, −/−) compared with wild-type mice (wt, +/+).

FIG. 3B is a graph showing the results a 15-hr overnight (ON) session in LABORAS® (Laboratory Animal Behaviour Observation Registration and Analysis System) for knockout mice (mutant, −/−) compared with wild-type mice (wt, +/+).

FIG. 4 shows immunohistological staining of GluR2 in the hippocampus of knockout mice (mutant, −/−) compared with wild-type mice (wt, +/+).

FIG. 5A shows that VDCC γ-8 knockout mice show decreased time in a chamber containing an intruder mouse, compared to wild-type mice;

FIG. 5b shows ten approach avoidance scores for VDCC γ-8 knockout and wild-type mice; and

FIG. 7 shows the location of the primers and the genomic structure of the regions of the VDCC γ-8 locus used in the targeting strategy.

### SEQUENCE LISTINGS

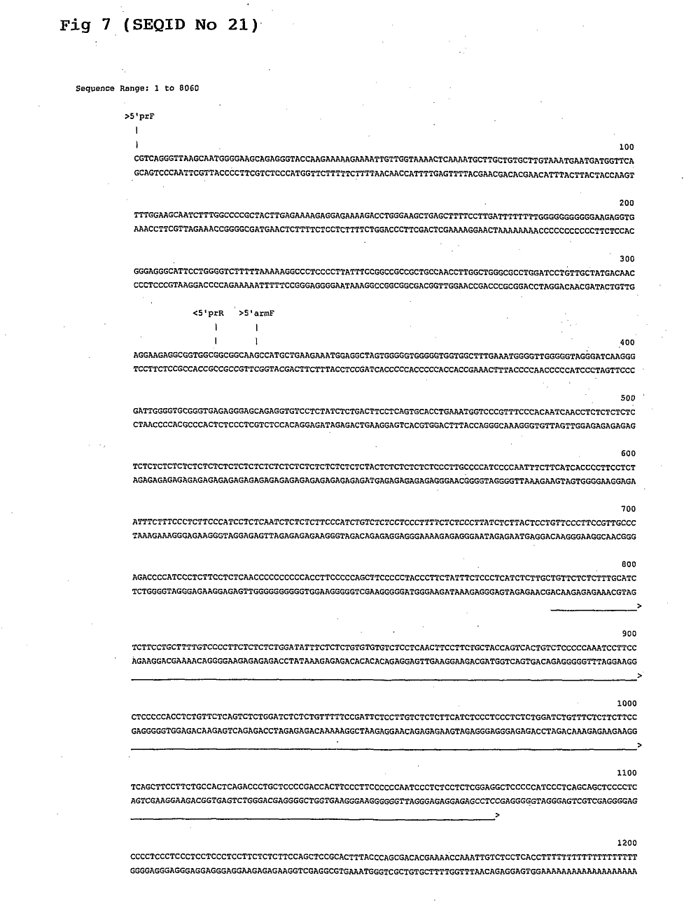

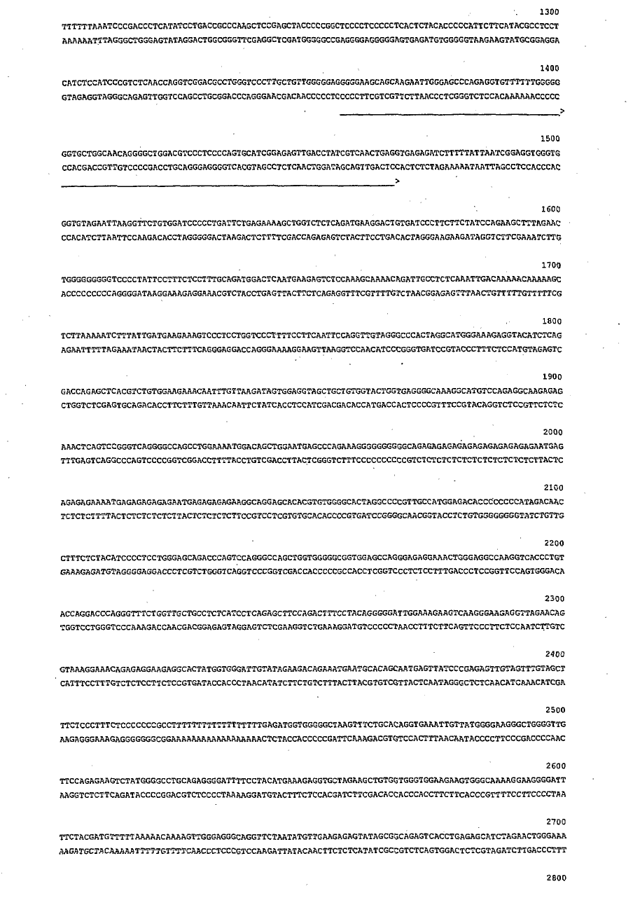

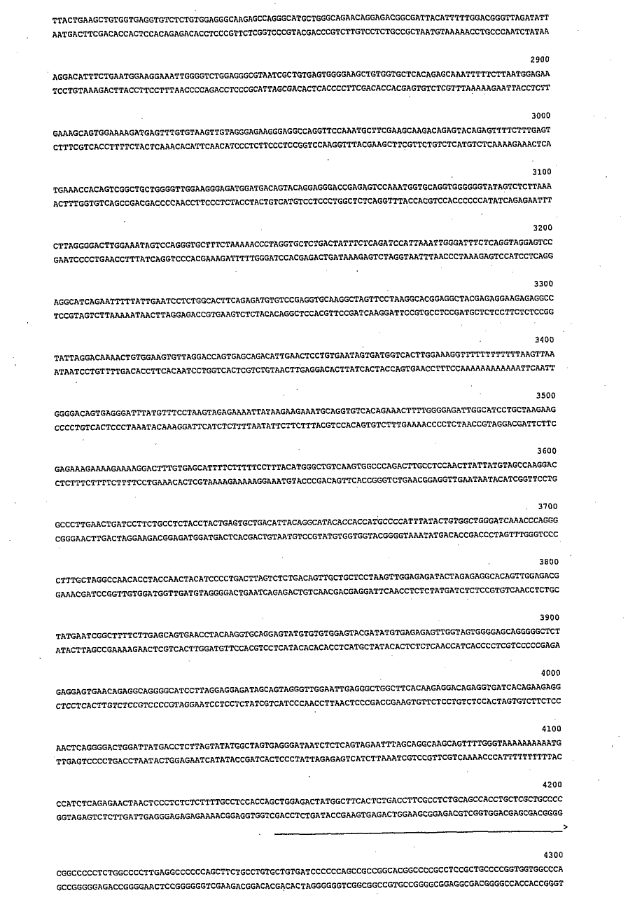

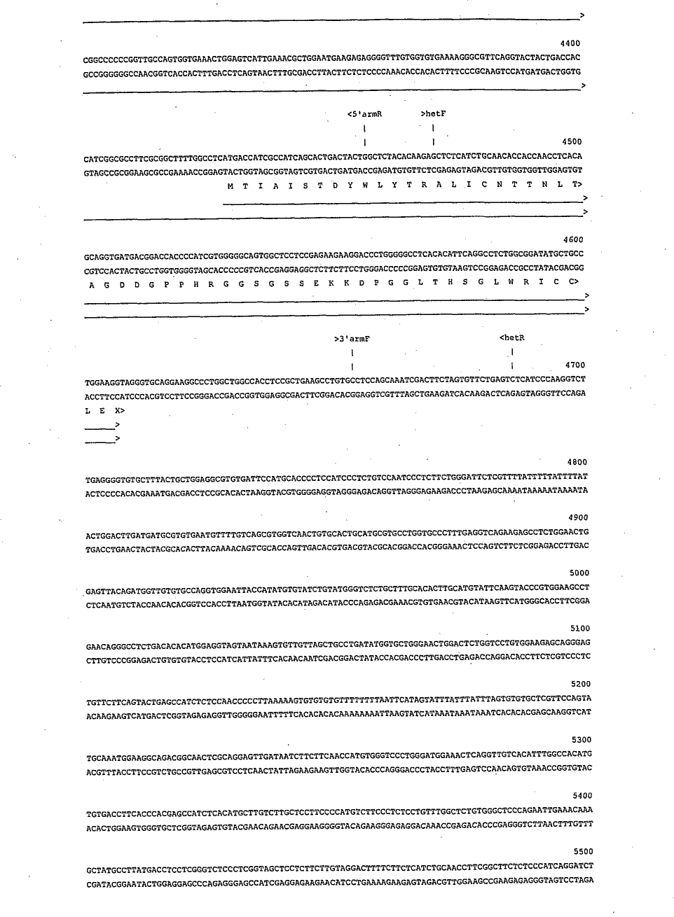

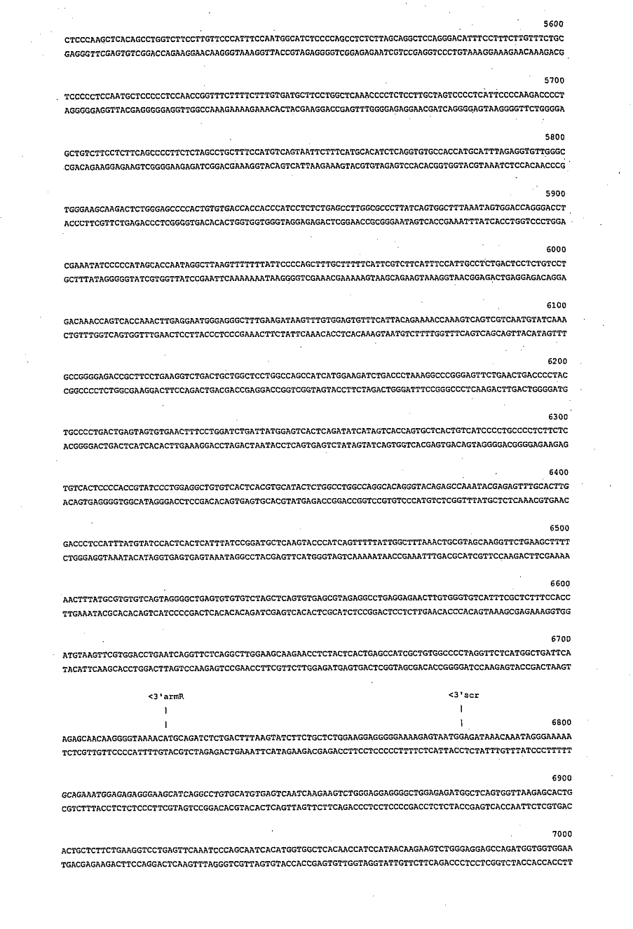

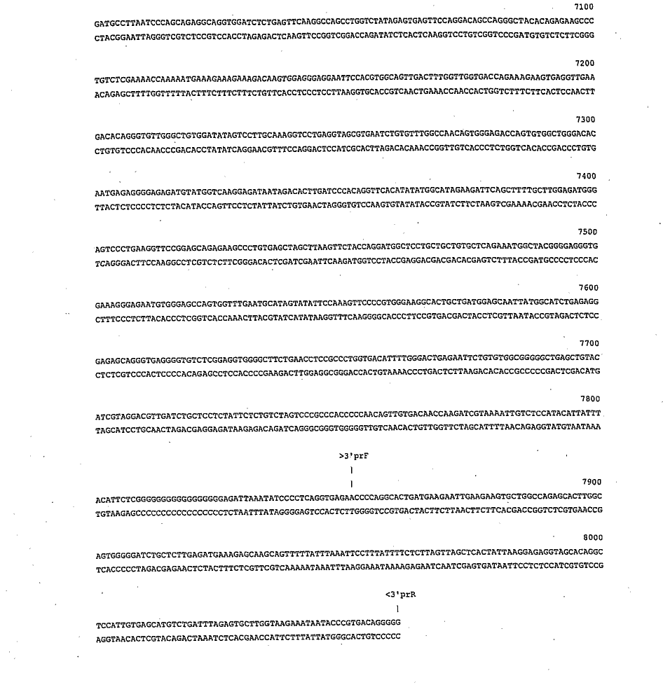

SEQ ID NO: 1 shows the cDNA sequence of human VDCC γ-8. SEQ ID NO: 2 shows an open reading frame derived from SEQ ID NO: 1. SEQ ID NO: 3 shows the amino acid sequence of human VDCC γ-8 derived from sequence 2. SEQ ID NO: 4 shows the open reading frame of a cDNA for Mouse VDCC γ-8. SEQ ID NO: 5 shows the amino acid sequence of Mouse VDCC γ-8. SEQ ID No. 6 shows an alternative open reading frame of a cDNA for human VDCC γ-8. SEQ ID No. 7 shows the amino acid sequence of human VDCC γ-8 derived from SEQ ID No. 6. SEQ ID No. 8-20 show the genotyping primers used to construct the knockout plasmid. SEQ ID No: 21 (FIG. 7) shows the location of the primers and the genomic structure of the regions of the VDCC γ-8 locus used in the targeting strategy.

### DETAILED DESCRIPTION

### VDCC γ-8 Ion Channel

The VDCC γ-8 ion channel, as well as homologues, variants or derivatives thereof, as well as its use in the treatment and diagnosis of VDCC γ-8 associated diseases, is described herein.

VDCC γ-8 is structurally related to other proteins of the ion channel family, as shown by the results of sequencing the amplified cDNA products encoding human VDCC γ-8. The cDNA sequence of SEQ ID NO: 1 contains two possible open reading frames: nucleotide numbers 104 to 1381 (shown as SEQ ID NO:2) encoding a polypeptide of 425 amino acids (shown as SEQ ID NO: 3) and nucleotide numbers 206 to 1381 (shown as SEQ ID NO:6) encoding polypeptide of 391 amino acids (shown as SEQ ID NO: 7). Human VDCC γ-8 is found to map to Homo sapiens chromosome Ch19q13.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press inc., Baca Raton, Fla., 1988, ISBN 0-8493-6078-1, John D. Pound (ed); “Immunochemical Protocols, vol 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3; and The Merck Manual of Diagnosis and Therapy (17th Edition, Beers, M. H., and Berkow, R, Eds, ISBN: 0911910107, John Wiley & Sons). Each of these general texts is herein incorporated by reference. Each of these general texts is herein incorporated by reference.

### Identities and Similarities to VDCC γ-8

Analysis of the VDCC γ-8 polypeptide (SEQ ID NO: 3) using the HMM structural prediction software of pfam (http://www.sanger.ac.uk/Software/Pfam/search.shtml) confirms that VDCC γ-8 peptide is an ion channel.

The mouse homologue of the human VDCC γ-8 ion channel has been cloned, and its nucleic acid sequence and amino acid sequence are shown as SEQ ID NO: 4 and SEQ ID NO: 5 respectively. The mouse VDCC γ-8 ion channel cDNA (SEQ ID NO: 4) shows a high degree of identity with the human VDCC γ-8 ion channel (SEQ ID NO: 2 and SEQ ID NO: 6) sequence, while the amino acid sequence (SEQ ID NO: 5) of the mouse VDCC γ-8 ion channel shows a high degree of identity and similarity with the human VDCC γ-8 ion channel (SEQ ID NO: 3 and SEQ ID NO: 7).

Human and mouse VDCC γ-8 ion channel are therefore members of a large family of ion channels.

### Expression Profile of VDCC γ-8

Polymerase chain reaction (PCR) amplification of VDCC γ-8 cDNA detects expression of VDCC γ-8 to varying abundance in the hippocampus (+++), cerebellum, cortex, striatum, mid brain, pons (++), hypothalamus, thalamus, forebrain, spinal cord, pituitary, and trigeminal ganglion (+).

LacZ staining in VDCC γ-8 knockout mice reveals strong expression of VDCC γ-8 in the brain and in particular in the hippocampus, cortex, amygdala, olfactory cortex, forebrain and thalamus.

Using VDCC γ-8 cDNA to search the human or mouse EST data sources by BLASTN, identities are found in cDNA libraries. This indicates that VDCC γ-8 is expressed in these normal or abnormal tissues:
Accession Numbers CX223421, DN176885, CX205409: Lateral wall of lateral ventricle
Accession Numbers BE647856, BE864111 Mouse brain (from a mix of cerebellum, brain stems, olfactory bulbs, hypothalamus, cortex, amygdala, basal ganglia, pineal gland, striatum, hipoccampus)
Accession Number BB641732: Neonate cortex
Accession Number BG803929: Neural retina

Accordingly, the VDCC γ-8 polypeptides, nucleic acids, probes, antibodies, expression vectors and ligands are useful for detection, diagnosis, treatment and other assays for diseases associated with over-, under- and abnormal expression of VDCC γ-8 ion channel in these and other tissues. Preferably, the diseases include the VDCC γ-8 associated diseases set out below.

### VDCC γ-8 Ion Channel Associated Diseases

According to the methods and compositions described here, VDCC γ-8 ion channel is useful in therapy and diagnosis, for treating and diagnosing a range of diseases. VDCC γ-8 is also useful in assaying molecules that modify the activity of VDCC γ-8, as discussed below. A molecule that modifies VDCC γ-8 is a potential therapeutic agent for a VDCC γ-8 associated disease. Diseases in which VDCC γ-8 is implicated are referred to for convenience as VDCC γ-8 associated diseases. These diseases are mental illness. As used herein, the term mental illness refers to disorders of brain function, including illnesses that include affective or emotional instability, behavioural dysregulation and/or cognitive dysfunction or impairment. The term “mental illness” also includes disorders exhibiting seizures, for example epilepsy. Human and animal (veterinary) therapy and diagnosis is within the scope of the invention.

Human VDCC γ-8 maps to Homo sapiens chromosome 19q13. Accordingly, in a specific embodiment, Ch19q13 ion channel may be used to treat or diagnose a disease which maps to this locus, chromosomal band, region, arm or the same chromosome. Known diseases which have been determined as being linked to the same locus, chromosomal band, region, arm or chromosome as the chromosomal location of VDCC γ-8 ion channel (i.e., Homo sapiens chromosome 19q13) include generalized epilepsy with febrile seizures, anemia and diabetes.

Knockout mice deficient in VDCC γ-8 display a range of phenotypes, as demonstrated in the Examples. For example, in the LABORAS test, VDCC γ-8 knockout mice are hyperactive initially; suggesting slower habituation to the novel environment compared to wildtype control mice (Example 5), an indication of working memory deficits, which can be a symptom of altered synaptic organisation. We therefore disclose a method of altering synaptic plasticity in an individual, the method comprising modulating the level or activity of VDCC γ-8 in that individual. As noted elsewhere, this can be achieved by modulating the expression of VDCC γ-8, or by use of modulators of (such as agonists or antagonists) VDCC γ-8.

VDCC γ-8 and modulators of VDCC γ-8 activity, including in particular agonists of VDCC γ-8, may be used to treat or alleviate diseases or syndromes in which synaptic plasticity in the relevant brain regions feature. Such diseases include status epilepticus disease, generalised seizures and all disorders exhibiting seizures, childhood and developmental disorders such as autism, autistic spectrum of diseases, pervasive developmental disorder, disintegrative disorder, Asperger's syndrome, Rett syndrome, Attention-Deficit/Hyperactivity disorder (ADHD), cognitive disorders, including, but not exclusively Alzheimer's disease, ishaemic/vascular dementia, Pick's disease, diffuse Lewy body dementia, frontotemporal dementias, corticobasal degeneration, Huntington's disease, progressive supranuclear palsy, AIDS/HIV dementia, prion infections, encephalitis, neurosyphilis, vasculitis, progressive multifocal leukoencephalopathy, and psychoses, including schizophrenia, generalised anxiety disorder, social anxiety, post traumatic stress disorder, phobias, social phobia, special phobias, panic disorder, obsessive compulsive disorder, acute stress disorder, separation disorder and depression, major depression, dysthymia, bipolar disorder, seasonal affective disorder, post natal depression, acute neurodegenerative disease, stroke and traumatic brain injury.

In a preferred embodiment, the VDCC γ-8 associated disease comprises a disease in which stress or anxiety is a symptom.

In a further preferred embodiment, the VDCC γ-8 associated disease comprises a disease in which psychosis is, or can be, a symptom. Psychosis is generally considered to be a symptom of severe mental illness, but is not a diagnosis in itself. Any mental illness that is or can be associated with psychosis is within the scope of the invention. Preferred diseases involving psychosis are schizophrenia bipolar disorder (manic depression) and severe clinical depression. Brain injury (or other neurological disorder), drug intoxication and withdrawal (especially alcohol, barbiturates and benzodiazepenes), lupus, electrolyte disorders in the elderly (such as urinary tract infections), pain syndromes, sleep depression and extreme stress (such as post-traumatic stress disorder) can also cause a psychotic episode, and are within the scope of the invention.

In yet another preferred embodiment, the VDCC γ-8 associated disease is a disorder exhibiting seizures, including generalised epilepsy and status epilepticus disease.

The most preferred VDCC γ-8 associated diseases are psychosis, schizophrenia, ADHD, bipolar disorder, major depression, generalised anxiety disorder and epilepsies including status epilepticus.

The experimental data detailed herein strongly implicate VDCC γ-8 in schizophrenia. The hyperactivity and the reduction in expression of GluR2 receptor subunits in the hippocampus (Example 5) indicate strongly that the VDCCγ-8 KO mice show a forebrain glutamatergic deficit and symptoms equivalent to the negative aspects of schizophrenia. The ability to make a special map of a new environment is impaired in the KO mice (Example 5) resulting in an increased period of exploratory behaviour. This indicates a role for VDCCγ-8 in schizophrenia in line with the glutamatergic hypothesis of schizophrenia proposed by Carlsson (Carlsson A. 1988, The current status of the dopamine theory of schizophrenia. Neuropharmacology 1, 179-186).

The KO mice also display a lack of social interaction (Example 6). These mice do not display any changes in anxiety levels therefore the lack of social interaction suggests a phenotype with similarities to the negative symptoms of schizophrenia as described above. KO mice also show significant pre-pulse inhibition disruption (Example 7), which is very strong evidence that VDCCγ8 (TARPγ8) has utility in the therapy of schizophrenia.

The reduction in GluR2 expression, hyperactivity, lack of social interaction and the PPI deficit all include that the KO mice have a phenotype similar to that observed in wild type mice following the administration of glutamatergic NMDA receptor antagonists. These are known to be psychotomimetic in humans and are routinely used as chemical models of schizophrenia in academic research and drug discovery programmes. Therefore, in a preferred embodiment, the VDCC γ-8 associated disease is schizophrenia.

As noted above, VDCC γ-8 ion channel may be used to diagnose and/or treat any of these specific diseases using any of the methods and compositions described here.

In particular, the use of nucleic acids, vectors comprising VDCC γ-8 ion channel nucleic acids, polypeptides, including homologues, variants or derivatives thereof, pharmaceutical compositions, host cells, and transgenic animals comprising VDCC γ-8 ion channel nucleic acids and/or polypeptides, for the treatment or diagnosis of the specific diseases listed above, is intended. Furthermore, compounds capable of interacting with or binding to VDCC γ-8 ion channel, preferably antagonists of VDCC γ-8, preferably a compound capable increasing the conductance of the channel are within the scope of the invention, antibodies against VDCC γ-8 ion channel, as well as methods of making or identifying these, in diagnosis or treatment of the specific diseases mentioned above. In particular, the use of any of these compounds, compositions, molecules, etc, in the production of vaccines for treatment or prevention of the specific diseases is intended. Diagnostic kits for the detection of the specific diseases in an individual is also within the scope of the invention.

Methods of linkage mapping to identify such or further specific diseases treatable or diagnosable by use of VDCC γ-8 ion channel are known in the art, and are also described elsewhere in this document.

### Neuroexcitation, Seizure & Cognitive Deficits

Neuroexcitation, seizure and cognitive deficits, as well as disorders in which these are manifested, including transmembrane AMPA regulatory proteins (TARP) associated diseases, are well known in the art. A summary description follows:

Neuroexcitation can be defined as the electrical activation of cells in the brain; neuroexcitation is part of the normal functioning of the brain or can also be the result of abnormal activity related to malfunction, disease or injury.

Seizures are temporary alterations in brain function expressing themselves into a changed mental state, tonic or clonic movements and various other symptoms. They are due to temporary abnormal neuroexcitation of a group of brain cells. Seizures can result from a one-off brain insult (e.g. toxic, impact) or as a recurring symptom as a result of tissue damage/degeneration/malfunction.

Cognition can be defined as the operation of the mind by which one becomes aware of objects of thought or perception; it includes all aspects of perceiving, thinking, and remembering. A cognitive deficit is the disruption of this operation likely due to disrupted neuroexcitation as a result of malfunction, disease or injury.

The VDCC γ-8 ion channel modulators may be used to treat or alleviate any of these symptoms.

Lifestyle and diet can also contribute to altered neuroexcitability, seizure and/or cognition effects—for example: 1) exercise can reduce the rate of Alzheimer's disease onset (both amyloid load and learning deficits; Adlard et al. (2005) J. Neurosci. 25: 4217-21); 2) dietary omega-3 polyunsaturated fatty acid, docosahexaenoic acid (DHA) could be protective against beta-amyloid production, accumulation, and potential downstream toxicity (Lim et al. (2005) J Neurosci 25: 3032-40). We disclose the use of the VDCC γ-8 ion channel and its modulators in combination with such lifestyle manipulations to alleviate their neuroexcitability, seizure and/or cognition effects.

Certain drugs, both recreational and medicinal, can lead to changes in neuroexcitability, seizure and/or cognition as either side effects or withdrawal from the drug. Such drugs include caffeine, cannabis, alcohol, nicotine, cold remedies, antihistamines, cocaine, and amphetamine. We disclose the use of the VDCC γ-8 ion channel and its modulators in combination with such drugs to alleviate their neuroexcitability, seizure and/or cognition effects.

### VDCC γ-8 Ion Channel Polypeptides

As used here, the term “VDCC γ-8 ion channel polypeptide” is intended to refer to a polypeptide comprising the amino acid sequence shown in SEQ ID No. 3, SEQ ID NO: 5 or SEQ ID NO:7 or a homologue, variant or derivative thereof. Preferably, the polypeptide comprises or is a homologue, variant or derivative of the sequence shown in SEQ ID NO: 3.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

The terms “variant”, “homologue”, “derivative” or “fragment” as used in this document include any substitution of, variation of, modification of, replacement of, deletion of or addition of one (or more) amino acid from or to a sequence. Unless the context admits otherwise, references to “VDCC γ-8” and “VDCC γ-8 ion channel” include references to such variants, homologues, derivatives and fragments of VDCC γ-8.

Preferably, as applied to VDCC γ-8, the resultant amino acid sequence has ion channel activity, more preferably having at least the same activity of the VDCC γ-8 ion channel shown as SEQ ID NO: 3, SEQ ID NO: 5 or SEQ ID NO: 7. In particular, the term “homologue” covers identity with respect to structure and/or function providing the resultant amino acid sequence has ion channel activity. With respect to sequence identity (i.e. similarity), preferably there is at least 70%, more preferably at least 75%, more preferably at least 85%, even more preferably at least 90% sequence identity. More preferably there is at least 95%, more preferably at least 98%, sequence identity. These terms also encompass polypeptides derived from amino acids which are allelic variations of the VDCC γ-8 ion channel nucleic acid sequence.

Where reference is made to the “channel activity” or “biological activity” of an ion channel such as VDCC γ-8 ion channel, these terms are intended to refer to the metabolic or physiological function of the VDCC γ-8 ion channel, including similar activities or improved activities or these activities with decreased undesirable side effects. Also included are antigenic and immunogenic activities of the VDCC γ-8 ion channel. Examples of ion channel activity, and methods of assaying and quantifying these activities, are known in the art.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

VDCC γ-8 polypeptides as described here may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

VDCC γ-8 polypeptides may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of the polypeptide and/or which facilitate identification, extraction and/or purification of the polypeptides. Another heterologous sequence that is particularly preferred is a polyamino acid sequence such as polyhistidine which is preferably N-terminal. A polyhistidine sequence of at least 10 amino acids, preferably at least 17 amino acids but fewer than 50 amino acids is especially preferred.

The VDCC γ-8 ion channel polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

VDCC γ-8 polypeptides are advantageously made by recombinant means, using known techniques. However they may also be made by synthetic means using techniques well known to skilled persons such as solid phase synthesis. The polypeptides described here may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. Preferably the fusion protein will not hinder the function of the protein of interest sequence.

VDCC γ-8 polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the VDCC γ-8 ion channel protein may be mixed with carriers or diluents which will not interfere with the intended purpose of the protein and still be regarded as substantially isolated. A VDCC γ-8 polypeptide may also be in a substantially purified form, in which case it will generally comprise the protein in a preparation in which more than 90%, for example, 95%, 98% or 99% of the protein in the preparation is a VDCC γ-8 polypeptide.

We further describe peptides comprising a portion of a VDCC γ-8 polypeptide. Thus, fragments of VDCC γ-8 ion channel and its homologues, variants or derivatives are included. The peptides may be between 2 and 200 amino acids, preferably between 4 and 40 amino acids in length. The peptide may be derived from a VDCC γ-8 polypeptide as disclosed here, for example by digestion with a suitable enzyme, such as trypsin. Alternatively the peptide, fragment, etc may be made by recombinant means, or synthesised synthetically,

The term “peptide” includes the various synthetic peptide variations known in the art, such as a retroinverso D peptides. The peptide may be an antigenic determinant and/or a T-cell epitope. The peptide may be immunogenic in vivo. Preferably the peptide is capable of inducing neutralising antibodies in vivo.

By aligning VDCC γ-8 ion channel sequences from different species, it is possible to determine which regions of the amino acid sequence are conserved between different species (“homologous regions”), and which regions vary between the different species (“heterologous regions”).

The VDCC γ-8 polypeptides may therefore comprise a sequence which corresponds to at least part of a homologous region. A homologous region shows a high degree of homology between at least two species. For example, the homologous region may show at least 70%, preferably at least 80%, more preferably at least 90%, even more preferably at least 95% for example 98 or 99% identity at the amino acid level using the tests described above. Peptides which comprise a sequence which corresponds to a homologous region may be used in therapeutic strategies as explained in further detail below. Alternatively, the VDCC γ-8 ion channel peptide may comprise a sequence which corresponds to at least part of a heterologous region. A heterologous region shows a low degree of homology between at least two species.

### VDCC γ-8 Ion Channel Polynucleotides and Nucleic Acids

VDCC γ-8 polynucleotides, VDCC γ-8 nucleotides and VDCC γ-8 nucleic acids, methods of production and uses of these nucleic acids are intended.

The terms “VDCC γ-8 polynucleotide”, “VDCC γ-8 nucleotide” and “VDCC γ-8 nucleic acid” may be used interchangeably, and are intended to refer to a polynucleotide/nucleic acid comprising a nucleic acid sequence as shown in SEQ ID NO: 1, SEQ ID NO: 2, SEQ ID NO: 4 or SEQ ID NO: 6 or a homologue, variant or derivative thereof. Preferably, the polynucleotide/nucleic acid comprises or is a homologue, variant or derivative of the nucleic acid sequence SEQ ID NO: 1, SEQ ID NO: 2 or SEQ ID NO: 6, most preferably, SEQ ID NO: 2.

These terms are also intended to include a nucleic acid sequence capable of encoding a polypeptide and/or a peptide, i.e., a VDCC γ-8 polypeptide. Thus, VDCC γ-8 ion channel polynucleotides and nucleic acids comprise a nucleotide sequence capable of encoding a polypeptide comprising the amino acid sequence shown in SEQ ID NO: 3, SEQ ID NO: 5 or SEQ ID NO: 7, or a homologue, variant or derivative thereof. Preferably, the VDCC γ-8 ion channel polynucleotides and nucleic acids comprise a nucleotide sequence capable of encoding a polypeptide comprising the amino acid sequence shown in SEQ ID NO: 3, or a homologue, variant or derivative thereof.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

Preferably, the term “nucleotide sequence” means DNA.

The terms “variant”, “homologue”, “derivative” or “fragment” as used in this document include any substitution of, variation of, modification of, replacement of, deletion of or addition of one (or more) nucleic acids from or to the sequence of a VDCC γ-8 nucleotide sequence. Unless the context admits otherwise, references to “VDCC γ-8” and “VDCC γ-8 ion channel” include references to such variants, homologues, derivatives and fragments of VDCC γ-8.

Preferably, the resultant nucleotide sequence encodes a polypeptide having ion channel activity, preferably having at least the same activity of the ion channel shown as SEQ ID NO: 3, SEQ ID NO: 5 or SEQ ID NO: 7. Preferably, the term “homologue” is intended to cover identity with respect to structure and/or function such that the resultant nucleotide sequence encodes a polypeptide which has ion channel activity. With respect to sequence identity (i.e. similarity), preferably there is at least 70%, more preferably at least 75%, more preferably at least 85%, more preferably at least 90% sequence identity. More preferably there is at least 95%, more preferably at least 98% or 99%, sequence identity. These terms also encompass allelic variations of the sequences.

### Calculation of Sequence Homology

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol. 403-410) and the GENEWORKS suite of comparison tools. Both BLAST and FASTA are available for offline and online searching (Ausubel et al., 1999 ibid, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at http://www.ncbi.nih.gov/BLAST/blast_help.html, which is incorporated herein by reference. Search parameters can be defined and can be advantageously set over the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

Most preferably, sequence comparisons are conducted using the simple BLAST search algorithm provided at http://www.ncbi.nlm.nih.gov/BLAST. In some embodiments, no gap penalties are used when determining sequence identity.

### Hybridisation

Nucleotide sequences that are capable of hybridising to the sequences presented herein, or any fragment or derivative thereof, or to the complement of any of the above are disclosed.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Nucleotide sequences capable of selectively hybridising to the nucleotide sequences presented herein, or to their complement, will be generally at least 70%, preferably at least 75%, more preferably at least 85 or 90% and even more preferably at least 95%, 98% or 99%, homologous to the corresponding nucleotide sequences presented herein over a region of at least 20, preferably at least 25 or 30, for instance at least 40, 60 or 100 or more contiguous nucleotides. Preferred nucleotide sequences will comprise regions homologous to SEQ ID NO: 1, 2, 4 or 6 preferably at least 70%, 80% or 90% and more preferably at least 95% homologous to one of the sequences.

The term “selectively hybridizable” means that the nucleotide sequence used as a probe is used under conditions where a target nucleotide sequence is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with 32P.

Also included are nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred embodiment, nucleotide sequences are disclosed that can hybridise to one or more of the VDCC γ-8 ion channel nucleotide sequences under stringent conditions (e.g. 65° C. and 0.1×SSC {1×SSC=0.15 M NaCl, 0.015 M Na3 Citrate pH 7.0). Where the nucleotide sequence is double-stranded, both strands of the duplex, either individually or in combination, are encompassed. Where the nucleotide sequence is single-stranded, it is to be understood that the complementary sequence of that nucleotide sequence is also of use.

Nucleotide sequences that are within the scope of the invention are capable of hybridising to the sequences that are complementary to the sequences presented herein, or any fragment or derivative thereof. Likewise, the invention encompasses nucleotide sequences that are complementary to sequences that are capable of hybridising to the sequence. These types of nucleotide sequences are examples of variant nucleotide sequences. In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (eg. 65° C. and 0.1×SSC {1×SSC=0.15 M NaCl, 0.015 Na3 citrate pH 7.0}) to the nucleotide sequences presented herein.

### Cloning of VDCC γ-8 Ion Channel and Homologues

The present invention encompasses nucleotide sequences that are complementary to the sequences presented here, or any fragment or derivative thereof. If the sequence is complementary to a fragment thereof then that sequence can be used as a probe to identify and clone similar ion channel sequences in other organisms etc.

This enables the cloning of VDCC γ-8 ion channel, its homologues and other structurally or functionally related genes from human and other species such as mouse, pig, sheep, etc to be accomplished. Polynucleotides which are identical or sufficiently identical to a nucleotide sequence contained in SEQ ID NO: 1, SEQ ID NO: 2, SEQ ID NO: 4, SEQ ID NO: 6 or a fragment thereof, may be used as hybridization probes for cDNA and genomic DNA, to isolate partial or full-length cDNAs and genomic clones encoding VDCC γ-8 ion channel from appropriate libraries. Such probes may also be used to isolate cDNA and genomic clones of other genes (including genes encoding homologues and orthologues from species other than human) that have sequence similarity, preferably high sequence similarity, to the VDCC γ-8 ion channel gene. Hybridization screening, cloning and sequencing techniques are known to those of skill in the art and are described in, for example, Sambrook et al (supra).

Typically nucleotide sequences suitable for use as probes are 70% identical, preferably 80% identical, more preferably 90% identical, even more preferably 95% identical to that of the referent. The probes generally will comprise at least 15 nucleotides. Preferably, such probes will have at least 30 nucleotides and may have at least 50 nucleotides. Particularly preferred probes will range between 150 and 500 nucleotides, more particularly about 300 nucleotides.

In one embodiment, to obtain a polynucleotide encoding a VDCC γ-8 polypeptide, including homologues and orthologues from species other than human, comprises the steps of screening an appropriate library under stringent hybridization conditions with a labelled probe having the SEQ ID NO: 1, SEQ ID NO: 2, SEQ ID NO: 4, SEQ ID NO: 6 or a fragment thereof and isolating partial or full-length cDNA and genomic clones containing said polynucleotide sequence. Such hybridization techniques are well known to those of skill in the art. Stringent hybridization conditions are as defined above or alternatively conditions under overnight incubation at 42 degrees C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 microgram/ml denatured, sheared salmon sperm DNA, followed by washing the filters in 0.1×SSC at about 65 degrees C.

### Functional Assay for VDCC γ-8 Ion Channel

The cloned putative VDCC γ-8 ion channel polynucleotide may be verified by sequence analysis or functional assays. For example, the putative VDCC γ-8 ion channel or homologue may be assayed for activity as follows. Capped RNA transcripts from linearized plasmid templates encoding the VDCC γ-8 receptor cDNAs are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected, co-expressed with glutamate receptors or other VOCC subunits in a 50 nl bolus using a microinjection apparatus. Two electrode voltage clamps are used to measure the currents from individual Xenopus oocytes in response to agonist exposure. Recordings are made in ND96 solution medium at room temperature. The Xenopus system may also be used to screen known ligands and tissue/cell extracts for activating ligands, as described in further detail below. Alternative functional assays include whole cell electrophysiology, fluorescence resonance energy transfer (FRET) analysis and FLIPR analysis.

### Cell Surface Expression Assay for VDCCγ-8 Ion-Channel

Stage V defolliculated oocytes are obtained, and RNA transcripts (10 ng/oocyte) are injected, co-expressed with glutamate receptors (such as GluR1-flip) into which a haemaglutinin epitope has been engineered into the extracellular region in a 50 nl bolus using a microinjection apparatus. 3 days after injection oocytes are incubated for 1 h with 0.25 μg/ml rat ant-HA antibody (3F10, Roche) followed by 30 min with horseradish-peroxidase-conjugated anti-rat immunoglobulin. Individual oocytes are placed into 50 μl of SuperSignal ELISA Femto maximum sensitivity substrate (Pierce) and quantified by chemiluminescence using a suitable luminometer such as a TD20/20m (Turner Designs).

### Expression Assays for VDCC γ-8 Ion Channel

In order to design useful therapeutics for treating VDCC γ-8 ion channel associated diseases, it is useful to determine the expression profile of VDCC γ-8 (whether wild-type or a particular mutant). Thus, methods known in the art may be used to determine the organs, tissues and cell types (as well as the developmental stages) in which VDCC γ-8 is expressed. For example, traditional or “electronic” Northerns may be conducted. Reverse-transcriptase PCR(RT-PCR) may also be employed to assay expression of the VDCC γ-8 gene or mutant. More sensitive methods for determining the expression profile of VDCC γ-8 include RNAse protection assays, as known in the art.

Northern analysis is a laboratory technique used to detect the presence of a transcript of a gene and involves the hybridization of a labelled nucleotide sequence to a membrane on which RNAs from a particular cell type or tissue have been bound. (Sambrook, supra, ch. 7 and Ausubel, F. M. et al. supra, ch. 4 and 16). Analogous computer techniques (“electronic Northerns”) applying BLAST may be used to search for identical or related molecules in nucleotide databases such as GenBank or the LIFESEQ database (Incyte Pharmaceuticals). This type of analysis has advantages in that they may be faster than multiple membrane-based hybridizations. In addition, the sensitivity of the computer search can be modified to determine whether any particular match is categorized as exact or homologous.

The polynucleotides and polypeptides described here, including the probes described above, may be employed as research reagents and materials for discovery of treatments and diagnostics to animal and human disease.

### Expression of VDCC γ-8 Ion Channel Polypeptides

A VDCC γ-8 polypeptide can be produced by (in general terms) culturing a host cell comprising a nucleic acid encoding VDCC γ-8 ion channel polypeptide, or a homologue, variant, or derivative thereof, under suitable conditions (i.e., conditions in which the VDCC γ-8 ion channel polypeptide is expressed), as will be apparent to one skilled in the art.

In order to express a biologically active VDCC γ-8 ion channel, the nucleotide sequences encoding VDCC γ-8 ion channel or homologues, variants, or derivatives thereof are inserted into appropriate expression vector, i.e., a vector which contains the necessary elements for the transcription and translation of the inserted coding sequence.

Methods which are well known to those skilled in the art are used to construct expression vectors containing sequences encoding VDCC γ-8 ion channel and appropriate transcriptional and translational control elements. These methods include in vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized to contain and express sequences encoding VDCC γ-8 ion channel. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The nature of the host cell employed does not matter.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (GIBCO/BRL), and the like, may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding VDCC γ-8 ion channel, vectors based on SV40 or EBV may be used with an appropriate selectable marker.

In bacterial systems, a number of expression vectors may be selected depending upon the use intended for VDCC γ-8 ion channel. For example, when large quantities of VDCC γ-8 ion channel are needed for the induction of antibodies, vectors which direct high level expression of fusion proteins that are readily purified may be used. Such vectors include, but are not limited to, multifunctional E. coli cloning and expression vectors such as BLUESCRIPT (Stratagene), in which the sequence encoding VDCC γ-8 ion channel may be ligated into the vector in frame with sequences for the amino-terminal Met and the subsequent 7 residues of β-galactosidase so that a hybrid protein is produced, pIN vectors (Van Heeke, G. and S. M. Schuster (1989) J. Biol. Chem. 264:5503-5509), and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. For reviews, see Ausubel (supra) and Grant et al. (1987; Methods Enzymol. 153:516-544).

In cases where plant expression vectors are used, the expression of sequences encoding VDCC γ-8 ion channel may be driven by any of a number of promoters. For example, viral promoters such as the 35S and 19S promoters of CaMV may be used alone or in combination with the omega leader sequence from TMV. (Takamatsu, N. (1987) EMBO J. 6:307-311). Alternatively, plant promoters such as the small subunit of RUBISCO or heat shock promoters may be used. (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews. (See, for example, Hobbs, S. or Murry, L. E. in McGraw Hill Yearbook of Science and Technology (1992) McGraw Hill, New York, N.Y.; pp. 191-196).

An insect system may also be used to express VDCC γ-8 ion channel. For example, in one such system, Autographa californica nuclear polyhedrosis virus (AcNPV) is used as a vector to express foreign genes in Spodoptera frugiperda cells or in Trichoplusia larvae. The sequences encoding VDCC γ-8 ion channel may be cloned into a non-essential region of the virus, such as the polyhedrin gene, and placed under control of the polyhedrin promoter. Successful insertion of VDCC γ-8 ion channel will render the polyhedrin gene inactive and produce recombinant virus lacking coat protein. The recombinant viruses may then be used to infect, for example, S. frugiperda cells or Trichoplusia larvae in which TrpM8 ion channel may be expressed. (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding VDCC γ-8 ion channel may be ligated into an adenovirus transcription/translation complex consisting of the late promoter and tripartite leader sequence. Insertion in a non-essential E1 or E3 region of the viral genome may be used to obtain a viable virus which is capable of expressing VDCC γ-8 ion channel in infected host cells. (Logan, J. and T. Shenk (1984) Proc. Natl. Acad. Sci. 81:3655-3659). In addition, transcription enhancers, such as the Rous sarcoma virus (RSV) enhancer, may be used to increase expression in mammalian host cells.

Thus, for example, the VDCC γ-8 receptors may be expressed in either human embryonic kidney 293 (HEK293) cells or adherent CHO cells. To maximize receptor expression, typically all 5′ and 3′ untranslated regions (UTRs) are removed from the receptor cDNA prior to insertion into a pCDN or pcDNA3 vector. The cells are transfected with individual receptor cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual receptors, about 24 clones are typically selected and analyzed by Northern blot analysis. Receptor mRNAs are generally detectable in about 50% of the G418-resistant clones analyzed.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding VDCC γ-8 ion channel. Such signals include the ATG initiation codon and adjacent sequences. In cases where sequences encoding VDCC γ-8 ion channel and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162).

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of stably expressing VDCC γ-8 ion channel can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tk- or apr-cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G-418 (Colbere-Garapin, F. et al (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51). Recently, the use of visible markers has gained popularity with such markers as anthocyanins, β-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131).

Although the presence/absence of marker gene expression suggests that the gene of interest is also present, the presence and expression of the gene may need to be confirmed. For example, if the sequence encoding VDCC γ-8 ion channel is inserted within a marker gene sequence, transformed cells containing sequences encoding VDCC γ-8 ion channel can be identified by the absence of marker gene function. Alternatively, a marker gene can be placed in tandem with a sequence encoding VDCC γ-8 ion channel under the control of a single promoter. Expression of the marker gene in response to induction or selection usually indicates expression of the tandem gene as well.

Alternatively, host cells which contain the nucleic acid sequence encoding VDCC γ-8 ion channel and express VDCC γ-8 ion channel may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

The presence of polynucleotide sequences encoding VDCC γ-8 ion channel can be detected by DNA-DNA or DNA-RNA hybridization or amplification using probes or fragments or fragments of polynucleotides encoding VDCC γ-8 ion channel. Nucleic acid amplification based assays involve the use of oligonucleotides or oligomers based on the sequences encoding VDCC γ-8 ion channel to detect transformants containing DNA or RNA encoding VDCC γ-8 ion channel.

A variety of protocols for detecting and measuring the expression of VDCC γ-8 ion channel, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on VDCC γ-8 ion channel is preferred, but a competitive binding assay may be employed. These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding VDCC γ-8 ion channel include oligolabeling, nick translation, end-labeling, or PCR amplification using a labeled nucleotide. Alternatively, the sequences encoding VDCC γ-8 ion channel, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), GE Healthcare (UK) and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding VDCC γ-8 subunits may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode VDCC γ-8 subunits may be designed to contain signal sequences which direct secretion of VDCC γ-8 subunits through a prokaryotic or eukaryotic cell membrane. Other constructions may be used to join sequences encoding VDCC γ-8 subunit to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the VDCC γ-8 subunit encoding sequence may be used to facilitate purification. One such expression vector provides for expression of a fusion protein containing VDCC γ-8 subunit and a nucleic acid encoding 6 histidine residues preceding a thioredoxin or an enterokinase cleavage site. The histidine residues facilitate purification on immobilized metal ion affinity chromatography (IMIAC; described in Porath, J. et al. (1992) Prot. Exp. Purif. 3: 263-281), while the enterokinase cleavage site provides a means for purifying VDCC γ-8 subunits from the fusion protein. A discussion of vectors which contain fusion proteins is provided in Kroll, D. J. et al. (1993; DNA Cell Biol. 12:441-453).

Fragments of VDCC γ-8 subunits may be produced not only by recombinant production, but also by direct peptide synthesis using solid-phase techniques. (Merrifield J. (1963) J. Am. Chem. Soc. 85:2149-2154). Protein synthesis may be performed by manual techniques or by automation. Automated synthesis may be achieved, for example, using the Applied Biosystems 431A peptide synthesizer (Perkin Elmer). Various fragments of VDCC γ-8 subunits may be synthesized separately and then combined to produce the full length molecule.

### Biosensors

The VDCC γ-8 polypeptides, nucleic acids, probes, antibodies, expression vectors and ligands are useful as (and for the production of) biosensors.

According to Aizawa (1988), Anal. Chem. Symp. 17: 683, a biosensor is defined as being a unique combination of a receptor for molecular recognition, for example a selective layer with immobilized antibodies or receptors such as a VDCC γ-8, and a transducer for transmitting the values measured. One group of such biosensors will detect the change that is caused in the optical properties of a surface layer due to the interaction of the receptor with the surrounding medium. Among such techniques may be mentioned especially ellipsometry and surface plasmon resonance. Biosensors incorporating VDCC γ-8 may be used to detect the presence or level of VDCC γ-8 ligands more preferably with regard to the diseases defined above. The construction of such biosensors is well known in the art.

Thus, cell lines expressing VDCC γ-8 subunits may be used as reporter systems for detection of ligands such as ATP via receptor-promoted formation of [3H]inositol phosphates or other second messengers (Watt et al., 1998, J Biol Chem May 29; 273 (22):14053-8). Receptor-ligand biosensors are also described in Hoffman et al., 2000, Proc Natl Acad Sci USA October 10; 97 (21):11215-20. Optical and other biosensors comprising VDCC γ-8 may also be used to detect the level or presence of interaction with G-proteins and other proteins, as described by, for example, Figler et al, 1997, Biochemistry December 23; 36 (51):16288-99 and Sarrio et al., 2000, Mol Cell Biol 2000 July; 20 (14):5164-74). Sensor units for biosensors are described in, for example, U.S. Pat. No. 5,492,840. The skilled person will recognise equally the value of an antagonist of VDCC γ-8. For example, in an alternative embodiment, treatment ADHD may be effected by a VDCC γ-8 antagonist.

### Screening Assays

The VDCC γ-8 polypeptide, including homologues, variants, and derivatives, whether natural or recombinant, may be employed in a screening process for compounds which bind the subunit and which activate (agonists) or inhibit activation of (antagonists) of VDCC γ-8, alter the kinetics of associated channels or change expression thereof. Thus, such polypeptides may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1 (2):Chapter 5 (1991).

VDCC γ-8 ion channel polypeptides are responsible for many biological functions and are associated with the diseases listed above. Accordingly, it is desirous to find compounds and drugs which stimulate VDCC γ-8 ion channels on the one hand and Which can inhibit the function of VDCC γ-8 ion channels on the other hand. In general, agonists and antagonists are employed for therapeutic and prophylactic purposes for such conditions as excitotoxicity and cognitive deficits. The skilled person will understand that the data herein indicated that mice lacking VDCC γ-8 demonstrate a schizophrenic phenotype. Therefore, in a preferred embodiment, a compound or drug that activates VDCC γ-8 (i.e. an agonist) is a potential therapy for schizophrenia and other related mental illnesses, as indicated above. The skilled person will recognise equally the value of an antagonist of VDCC γ-8. For example, in an alternative embodiment, treatment of ADHD may be effected by a VDCC γ-8 antagonist.

Rational design of candidate compounds likely to be able to interact with VDCC γ-8 ion channel proteins may be based upon structural studies of the molecular shapes of a polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the VDCC γ-8 receptor polypeptide on the surface thereof. Such cells include cells from animals, yeast, Drosophila or E. coli. Cells expressing the receptor (or cell membrane containing the expressed receptor) are then contacted with a test compound to observe binding, or stimulation or inhibition of a functional response. For example, Xenopus oocytes may be injected with VDCC γ-8 mRNA or polypeptide, and currents induced by exposure to test compounds measured by use of voltage clamps measured, as described in further detail elsewhere.

Instead of testing each candidate compound individually with the VDCC γ-8 ion channel, a library or bank of candidate ligands may advantageously be produced and screened. Thus, for example, a bank of over 200 putative receptor ligands has been assembled for screening. The bank comprises: transmitters, hormones and chemokines known to act via an ion channel; naturally occurring compounds which may be putative agonists for an ion channel, non-mammalian, biologically active peptides for which a mammalian counterpart has not yet been identified; and compounds not found in nature, but which activate ion channels with unknown natural ligands. This bank is used to screen the receptor for known ligands, using both functional (i.e. calcium, microphysiometer, FLIPR assay, whole cell electrophysiology, oocyte electrophysiology, etc, see elsewhere) as well as binding assays as described in further detail elsewhere. However, a large number of mammalian receptors exist for which there remains, as yet, no cognate activating ligand (agonist) or deactivating ligand (antagonist). Thus, active ligands for these receptors may not be included within the ligands banks as identified to date. Accordingly, VDCC γ-8 may also be functionally screened (using calcium, cAMP, microphysiometer, oocyte electrophysiology, etc., functional screens) against tissue extracts to identify natural ligands. Extracts that produce positive functional responses can be sequentially subfractionated, with the fractions being assayed as described here, until an activating ligand is isolated and identified.

In such experiments, basal calcium levels in the HEK 293 cells in transfected or vector control cells are observed to be in the normal, 100 nM to 200 nM, range. HEK 293 cells expressing homomeric or heteromeric VDCC γ-8 ion channels or recombinant homomeric or heteromeric VDCC γ-8 ion channels are loaded with fura 2 and in a single day more than 150 selected ligands or tissue/cell extracts are evaluated for agonist induced calcium mobilization. Similarly, HEK 293 cells expressing VDCC γ-8 ion channel or recombinant VDCC γ-8 ion channel are evaluated for the increase or decrease of Ca2+ flux. Agonists presenting a calcium transient are tested in vector control cells to determine if the response is unique to the transfected cells expressing receptor.

Another method involves screening for receptor inhibitors by determining inhibition or stimulation of VDCC γ-8 ion channels. Such a method involves transfecting a eukaryotic cell with the VDCC γ-8 subunits either alone to form a homomeric channel or with other Trp channel subunits to form a heteromeric channel to express the receptor on the cell surface. The cell is then exposed to potential antagonists in the presence of the VDCC γ-8 receptor. The cell can be tested using whole cell electrophysiology to determine the changes in the conductance or kinetics of the current.

Another method for detecting agonists or antagonists of VDCC γ-8 is the yeast based technology as described in U.S. Pat. No. 5,482,835, incorporated by reference herein.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a VDCC γ-8 polypeptide or peptide are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are contacted or incubated with a labelled antibody known to bind to a VDCC γ-8 polypeptide, such as 125I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a VDCC γ-8 polypeptide, and is described in detail in International Patent application No. WO 84/03564 (Commonwealth Serum Labs.), published on Sep. 13, 1984. First, large numbers of different small peptide test compounds are synthesized on a solid substrate, e.g., plastic pins or some other appropriate surface; see Fodor et al. (1991). Then all the pins are reacted with solubilized VDCC γ-8 polypeptide and washed. The next step involves detecting bound polypeptide. Compounds which interact specifically with the polypeptide will thus be identified.

Ligand binding assays provide a direct method for ascertaining receptor pharmacology and are adaptable to a high throughput format. The purified ligand for a receptor may be radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing a VDCC γ-8 polypeptide to form a mixture, measuring VDCC γ-8 ion channel activity in the mixture, and comparing the VDCC γ-8 ion channel activity of the mixture to a standard.

The VDCC γ-8 subunit cDNA, protein and antibodies to the protein may also be used to configure assays for detecting the effect of added compounds on the production of VDCC γ-8 subunit mRNA and protein in cells. For example, an ELISA may be constructed for measuring secreted or cell associated levels of VDCC γ-8 subunit protein using monoclonal and polyclonal antibodies by standard methods known in the art, and this can be used to discover agents which may inhibit or enhance the production of VDCC γ-8 subunit (also called antagonist or agonist, respectively) from suitably manipulated cells or tissues. Standard methods for conducting screening assays are well understood in the art.

Examples of potential VDCC γ-8 ion channel antagonists and blockers include antibodies or, in some cases, nucleotides and their analogues, including purines and purine analogues, oligonucleotides or proteins which are closely related to the ligand of the VDCC γ-8 ion channel, e.g., a fragment of the ligand, or small molecules which bind to the receptor but do not elicit a response, so that the activity of the receptor is prevented.

We there therefore also provide a compound capable of binding specifically to a VDCC γ-8 polypeptide and/or peptide.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Preferably the compound is an antibody.

The materials necessary for such screening to be conducted may be packaged into a screening kit. Such a screening kit is useful for identifying agonists, antagonists, ligands, receptors, substrates, enzymes, etc. for VDCC γ-8 polypeptides or compounds which decrease or enhance the production of VDCC γ-8 ion channel polypeptides. The screening kit comprises: (a) a VDCC γ-8 polypeptide; (b) a recombinant cell expressing a VDCC γ-8 polypeptide; (c) a cell membrane expressing a VDCC γ-8 polypeptide; or (d) antibody to a VDCC γ-8 polypeptide. The screening kit may optionally comprise instructions for use.

### Transgenic Animals

Transgenic animals capable of expressing natural or recombinant VDCC γ-8 ion channel, or a homologue, variant or derivative, at elevated or reduced levels compared to the normal expression level are within the scope of the invention. Included are transgenic animals (“VDCC γ-8 knockout's) which do not express functional VDCC γ-8 receptor as a result of one or more loss of function mutations, including a deletion, of the VDCC γ-8 gene. Preferably, such a transgenic animal is a non-human mammal, such as a pig, a sheep or a rodent. Most preferably the transgenic animal is a mouse or a rat. Such transgenic animals may be used in screening procedures to identify agonists and/or antagonists of VDCC γ-8 ion channel, as well as to test for their efficacy as treatments for diseases in vivo.

In preferred embodiments, the transgenic VDCC γ-8 animals, particularly VDCC γ-8 knockouts, display the phenotypes set out in the Examples, preferably as measured by the tests set out therein. Thus, the VDCC γ-8 animals, particularly VDCC γ-8 knockouts, preferably display any one or more of the following: decreased rotarod performance, reduced habituation to a novel environment, cognitive deficits or seizure sensitivity.

In highly preferred embodiments, the VDCC γ-8 animals, particularly VDCC γ-8 knockouts, display at least 10%, preferably at least 20%, more preferably at least 30%, 40%, 50%, 60%, 70%, 80%, 90%, or 100% higher or lower (as the case may be) of the measured parameter as compared to the corresponding wild-type mice.

For example, transgenic animals that have been engineered to be deficient in the production of VDCC γ-8 ion channel may be used in assays to identify agonists and/or antagonists of VDCC γ-8 ion channel. One assay is designed to evaluate a potential drug (a candidate ligand or compound) to determine if it produces a physiological response in the absence of VDCC γ-8 ion channel receptors. This may be accomplished by administering the drug to a transgenic animal as discussed above, and then assaying the animal for a particular response. Although any physiological parameter could be measured in this assay, preferred responses include one or more of the following: changes to disease resistance; altered inflammatory responses; altered seizure sensitivity; altered cognitive performance; altered synaptic plasticity; altered social interaction.

Tissues derived from the VDCC γ-8 knockout animals may be used in receptor binding assays to determine whether the potential drug (a candidate ligand or compound) binds to the VDCC γ-8 receptor. Such assays can be conducted by obtaining a first receptor preparation from the transgenic animal engineered to be deficient in VDCC γ-8 receptor production and a second receptor preparation from a source known to bind any identified VDCC γ-8 ligands or compounds. In general, the first and second receptor preparations will be similar in all respects except for the source from which they are obtained. For example, if brain tissue from a transgenic animal (such as described above and below) is used in an assay, comparable brain tissue from a normal (wild type) animal is, used as the source of the second receptor preparation. Each of the receptor preparations is incubated with a ligand known to bind to VDCC γ-8 receptors, both alone and in the presence of the candidate ligand or compound. Preferably, the candidate ligand or compound will be examined at several different concentrations.

The extent to which binding by the known ligand is displaced by the test compound is determined for both the first and second receptor preparations. Tissues derived from transgenic animals may be used in assays directly or the tissues may be processed to isolate membranes or membrane proteins, which are themselves used in the assays. A preferred transgenic animal is the mouse. The ligand may be labeled using any means compatible with binding assays. This would include, without limitation, radioactive, enzymatic, fluorescent or chemiluminescent labeling (as well as other labelling techniques as described in further detail above).

Furthermore, antagonists of VDCC γ-8 ion channel receptor may be identified by administering candidate compounds, etc, to wild type animals expressing functional VDCC γ-8, and animals identified which exhibit any of the phenotypic characteristics associated with reduced or abolished expression of VDCC γ-8 receptor function.

Detailed methods for generating non-human transgenic animals are described in further detail below (Examples 1 and 2). Transgenic gene constructs can be introduced into the germ line of an animal to make a transgenic mammal. For example, one or several copies of the construct may be incorporated into the genome of a mammalian embryo by standard transgenic techniques.

In an exemplary embodiment, the transgenic non-human animals are produced by introducing transgenes into the germline of the non-human animal. Embryonal target cells at various developmental stages can be used to introduce transgenes. Different methods are used depending on the stage of development of the embryonal target cell. The specific line(s) of any animal are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the VDCC γ-8 receptor transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity may also be conducted. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this document, a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the methods described here will include exogenous genetic material. As set out above, the exogenous genetic material will, in certain embodiments, be a DNA sequence which results in the production of a VDCC γ-8 ion channel. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Non-human transgenic animals are provided, where the transgenic animal is characterized by having an altered VDCC γ-8 gene, preferably as described above, as models for VDCC γ-8 ion channel function. Alterations to the gene include deletions or other loss of function mutations, introduction of an exogenous gene having a nucleotide sequence with targeted or random mutations, introduction of an exogenous gene from another species, or a combination thereof. The transgenic animals may be either homozygous or heterozygous for the alteration. The animals and cells derived therefrom are useful for screening biologically active agents that may modulate VDCC γ-8 ion channel function. The screening methods are of particular use for determining the specificity and action of potential therapies for pain and cancer, particularly prostate cancer. The animals are useful as a model to investigate the role of VDCC γ-8 ion channels in normal tissues and organs such as the brain, heart, spleen and liver and the effect on their function.

Another aspect pertains to a transgenic nonhuman animal having a functionally disrupted endogenous VDCC γ-8 gene but which also carries in its genome, and expresses, a transgene encoding a heterologous VDCC γ-8 protein (i.e., a VDCC γ-8 from another species). Preferably, the animal is a mouse and the heterologous VDCC γ-8 is a human VDCC γ-8. An animal, or cell lines derived from such an animal, which has been reconstituted with human VDCC γ-8, can be used to identify agents that inhibit human VDCC γ-8 in vivo and in vitro. For example, a stimulus that induces signalling through human VDCC γ-8 can be administered to the animal, or cell line, in the presence and absence of an agent to be tested and the response in the animal, or cell line, can be measured. An agent that inhibits human VDCC γ-8 in vivo or in vitro can be identified based upon a decreased response in the presence of the agent compared to the response in the absence of the agent.

A VDCC γ-8 deficient transgenic non-human animal (a “VDCC γ-8 subunit knock-out”) is also provided. Such an animal is one which expresses lowered or no VDCC γ-8 ion channel activity, preferably as a result of an endogenous VDCC γ-8 ion channel genomic sequence being disrupted or deleted. Preferably, such an animal expresses no ion channel activity. More preferably, the animal expresses no activity of the VDCC γ-8 ion channel shown as SEQ ID NO: 3 or SEQ ID NO: 5. VDCC γ-8 ion channel knock-outs may be generated by various means known in the art, as described in further detail below.

The present invention also pertains to a nucleic acid construct for functionally disrupting a VDCC γ-8 gene in a host cell. The nucleic acid construct comprises: a) a non-homologous replacement portion; b) a first homology region located upstream of the non-homologous replacement portion, the first homology region having a nucleotide sequence with substantial identity to a first VDCC γ-8 gene sequence; and c) a second homology region located downstream of the non-homologous replacement portion, the second homology region having a nucleotide sequence with substantial identity to a second VDCC γ-8 gene sequence, the second VDCC γ-8 gene sequence having a location downstream of the first VDCC γ-8 gene sequence in a naturally occurring endogenous VDCC γ-8 gene. Additionally, the first and second homology regions are of sufficient length for homologous recombination between the nucleic acid construct and an endogenous VDCC γ-8 gene in a host cell when the nucleic acid molecule is introduced into the host cell. In a preferred embodiment, the non-homologous replacement portion comprises an expression reporter, preferably including lacZ and a positive selection expression cassette, preferably including a neomycin phosphotransferase gene operatively linked to a regulatory element(s).

Preferably, the first and second VDCC γ-8 gene sequences are derived from SEQ ID No. 1, SEQ ID No. 2, SEQ ID NO: 4 or SEQ ID NO: 6, or a homologue, variant or derivative thereof.

Another aspect pertains to recombinant vectors into which the nucleic acid construct described above has been incorporated. Yet another aspect pertains to host cells into which the nucleic acid construct has been introduced to thereby allow homologous recombination between the nucleic acid construct and an endogenous VDCC γ-8 gene of the host cell, resulting in functional disruption of the endogenous VDCC γ-8 gene. The host cell can be a mammalian cell that normally expresses VDCC γ-8 from the liver, brain, spleen or heart, or a pluripotent cell, such as a mouse embryonic stem cell. Further development of an embryonic stem cell into which the nucleic acid construct has been introduced and homologously recombined with the endogenous VDCC γ-8 gene produces a transgenic nonhuman animal having cells that are descendant from the embryonic stem cell and thus carry the VDCC γ-8 gene disruption in their genome. Animals that carry the VDCC γ-8 gene disruption in their germline can then be selected and bred to produce animals having the VDCC γ-8 gene disruption in all somatic and germ cells. Such mice can then be bred to homozygosity for the VDCC γ-8 gene disruption.

### Antibodies

For the purposes of this document, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. The antibodies and fragments thereof may be humanised antibodies, for example as described in EP-A-239400. Furthermore, antibodies with fully human variable regions (or their fragments), for example, as described in U.S. Pat. Nos. 5,545,807 and 6,075,181 may also be used. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A polypeptide or peptide of may be used to develop an antibody by known techniques. Such an antibody may be capable of binding specifically to the VDCC γ-8 ion channel protein or homologue, fragment, etc.

If polyclonal antibodies are desired, a selected mammal (e.g., mouse, rabbit, goat, horse, etc.) may be immunised with an immunogenic composition comprising a relevant polypeptide or peptide. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Serum from the immunised animal is collected and treated according to known procedures. If serum containing polyclonal antibodies to an epitope obtainable from a VDCC γ-8 polypeptide contains antibodies to other antigens, the polyclonal antibodies can be purified by immunoaffinity chromatography. Techniques for producing and processing polyclonal antisera are known in the art. In order that such antibodies may be made, we also provide amino acid sequences of VDCC γ-8 or fragments thereof haptenised to another amino acid sequence for use as immunogens in animals or humans.

Monoclonal antibodies directed against epitopes obtainable from a VDCC γ-8 ion channel polypeptide or peptide can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the trioma technique, the human B-cell hybridoma technique (Kosbor et al (1983) Immunol Today 4:72; Cote et al (1983) Proc Natl Acad Sci 80:2026-2030) and the EBV-hybridoma technique (Cole et al., Monoclonal Antibodies and Cancer Therapy, pp. 77-96, Alan R. Liss, Inc., 1985).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from a VDCC γ-8 ion channel polypeptide or peptide are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the polypeptide or peptide may also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse W D et al (1989) Science 256:1275-128 1).

Techniques for the production of single chain antibodies (U.S. Pat. No. 4,946,778) can also be adapted to produce single chain antibodies to VDCC γ-8 ion channel polypeptides. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against VDCC γ-8 ion channel polypeptides may also be employed to treat VDCC γ-8 associated diseases.

### Diagnostic Assays

This disclosure also relates to the use of VDCC γ-8 ion channel polynucleotides and polypeptides (as well as homologues, variants and derivatives thereof) for use in diagnosis as diagnostic reagents or in genetic analysis. Nucleic acids complementary to or capable of hybridising to VDCC γ-8 ion channel nucleic acids (including homologues, variants and derivatives), as well as antibodies against VDCC γ-8 polypeptides are also useful in such assays.

In a preferred embodiment, the diagnostic assay is carried out on a sample isolated from the patient, so that the assay is not carried out on a human or animal body.

Detection of a mutated form of the VDCC γ-8 ion channel gene associated with a dysfunction will provide a diagnostic tool that can add to or define a diagnosis of a disease or susceptibility to a disease which results from under-expression, over-expression or altered expression of VDCC γ-8 ion channel. Individuals carrying mutations in the VDCC γ-8 ion channel gene (including control sequences) may be detected at the DNA level by a variety of techniques.

For example, DNA may be isolated from a patient and the DNA polymorphism pattern of VDCC γ-8 determined. The identified pattern is compared to controls of patients known to be suffering from a disease associated with over-, under- or abnormal expression of VDCC γ-8. Patients expressing a genetic polymorphism pattern associated with VDCC γ-8 associated disease may then be identified. Genetic analysis of the VDCC γ-8 ion channel gene may be conducted by any technique known in the art. For example, individuals may be screened by determining DNA sequence of a VDCC γ-8 allele, by RFLP or SNP analysis, etc. Patients may be identified as having a genetic predisposition for a disease associated with the over-, under-, or abnormal expression of VDCC γ-8 by detecting the presence of a DNA polymorphism in the gene sequence for VDCC γ-8 or any sequence controlling its expression.

Patients so identified can then be treated to prevent the occurrence of VDCC γ-8 associated disease, or more aggressively in the early stages of VDCC γ-8 associated disease to prevent the further occurrence or development of the disease.

A kit for the identification of a patient's genetic polymorphism pattern associated with VDCC γ-8 associated disease is also provided. The kit includes DNA sample collecting means and means for determining a genetic polymorphism pattern, which is then compared to control samples to determine a patient's susceptibility to VDCC γ-8 associated disease. Kits for diagnosis of a VDCC γ-8 associated disease comprising VDCC γ-8 polypeptide and/or an antibody against such a polypeptide (or fragment of it) are also provided.

Nucleic acids for diagnosis may be obtained from a subject's cells, such as from blood, urine, saliva, tissue biopsy or autopsy material. In a preferred embodiment, the DNA is obtained from blood cells obtained from a finger prick of the patient with the blood collected on absorbent paper. In a further preferred embodiment, the blood will be collected on an AmpliCard.TM. (University of Sheffield, Department of Medicine and Pharmacology, Royal Hallamshire Hospital, Sheffield, England S10 2JF).

The DNA may be used directly for detection or may be amplified enzymatically by using PCR or other amplification techniques prior to analysis. Oligonucleotide DNA primers that target the specific polymorphic DNA region within the genes of interest may be prepared so that in the PCR reaction amplification of the target sequences is achieved. RNA or cDNA may also be used as templates in similar fashion. The amplified DNA sequences from the template DNA may then be analyzed using restriction enzymes to determine the genetic polymorphisms present in the amplified sequences and thereby provide a genetic polymorphism profile of the patient. Restriction fragments lengths may be identified by gel analysis. Alternatively, or in conjunction, techniques such as SNP (single nucleotide polymorphisms) analysis may be employed.

Deletions and insertions can be detected by a change in size of the amplified product in comparison to the normal genotype. Point mutations can be identified by hybridizing amplified DNA to labeled VDCC γ-8 ion channel nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, eg., Myers et al, Science (1985) 230:1242. Sequence changes at specific locations may also be revealed by nuclease protection assays, such as RNAse and S1 protection or the chemical cleavage method. See Cotton et al., Proc Natl Acad Sci USA (1985) 85: 4397-4401. In another embodiment, an array of oligonucleotides probes comprising the VDCC γ-8 ion channel nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

Single strand conformation polymorphism (SSCP) may be used to detect differences in electrophoretic mobility between mutant and wild type nucleic acids (Orita et al. (1989) Proc Natl. Acad. Sci. USA: 86:2766, see also Cotton (1993) Mutat Res 285:125-144; and Hayashi (1992) Genet Anal Tech Appl 9:73-79). Single-stranded DNA fragments of sample and control VDCC γ-8 nucleic acids may be denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

The presence of VDCC γ-8 polypeptides and nucleic acids may be detected in a sample. Thus, infections and diseases as listed above can be diagnosed by methods comprising determining from a sample derived from a subject an abnormally decreased or increased level of the VDCC γ-8 polypeptide or VDCC γ-8 ion channel mRNA. The sample may comprise a cell or tissue sample from an organism suffering or suspected to be suffering from a disease associated with increased, reduced or otherwise abnormal VDCC γ-8 expression, including spatial or temporal changes in level or pattern of expression. The level or pattern of expression of VDCC γ-8 in an organism suffering from or suspected to be suffering from such a disease may be usefully compared with the level or pattern of expression in a normal organism as a means of diagnosis of disease.

In general therefore, a method of detecting the presence of a nucleic acid comprising a VDCC γ-8 nucleic acid in a sample comprises contacting the sample with at least one nucleic acid probe which is specific for said nucleic acid and monitoring said sample for the presence of the nucleic acid. For example, the nucleic acid probe may specifically bind to the VDCC γ-8 ion channel subunit nucleic acid, or a portion of it, and binding between the two detected; the presence of the complex itself may also be detected. Furthermore, we encompass a method of detecting the presence of a VDCC γ-8 polypeptide by contacting a cell sample with an antibody capable of binding the polypeptide and monitoring said sample for the presence of the polypeptide. Monitoring the presence of a complex formed between the antibody and the polypeptide, or monitoring the binding between the polypeptide and the antibody may conveniently achieve this. Methods of detecting binding between two entities are known in the art, and include FRET (fluorescence resonance energy transfer), surface plasmon resonance, etc.

Decreased or increased expression can be measured at the RNA level using any of the methods well known in the art for the quantitation of polynucleotides, such as, for example, PCR, RT-PCR, RNAse protection, Northern blotting and other hybridization methods. Assay techniques that can be used to determine levels of a protein, such as VDCC γ-8, in a sample derived from a host are well-known to those of skill in the art. Such assay methods include radioimmunoassays, competitive-binding assays, Western Blot analysis and ELISA assays.

We further disclose a diagnostic kit for a disease or susceptibility to a disease (including an infection), for example, pain and cancer, particularly neuropathic pain and prostate cancer. The diagnostic kit comprises a VDCC γ-8 polynucleotide or a fragment thereof; a complementary nucleotide sequence; a VDCC γ-8 polypeptide or a fragment thereof, or an antibody to a VDCC γ-8 polypeptide.

### Chromosome Assays

The nucleotide sequences described here are also valuable for chromosome identification. The sequence is specifically targeted to and can hybridize with a particular location on an individual human chromosome. As described above, human VDCC γ-8 ion channel is found to map to Homo sapiens chromosome 19q13.

The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian heritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

### Prophylactic and Therapeutic Methods

Methods of treating an abnormal conditions related to both an excess of and insufficient amounts of VDCC γ-8 ion channel activity are provided.

If the activity of VDCC γ-8 ion channel is in excess, several approaches are available. One approach comprises administering to a subject an inhibitor compound (antagonist) as hereinabove described along with a pharmaceutically acceptable carrier in an amount effective to inhibit activation by blocking binding of ligands to the VDCC γ-8 ion channel, or by inhibiting a second signal, and thereby alleviating the abnormal condition.

In another approach, soluble forms of VDCC γ-8 polypeptides still capable of binding the ligand in competition with endogenous VDCC γ-8 ion channel may be administered. Typical embodiments of such competitors comprise fragments of the VDCC γ-8 polypeptide.

In still another approach, expression of the gene encoding endogenous VDCC γ-8 ion channel can be inhibited using expression blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of VDCC γ-8 ion channel and its activity, several approaches are also available. One approach comprises administering to a subject a therapeutically effective amount of a compound which activates VDCC γ-8 ion channel, i.e., an agonist as described above, in combination with a pharmaceutically acceptable carrier, to thereby alleviate the abnormal condition. Alternatively, gene therapy may be employed to effect the endogenous production of VDCC γ-8 ion channel by the relevant cells in the subject. For example, a VDCC γ-8 polynucleotide may be engineered for expression in a replication defective retroviral vector, as discussed above. The retroviral expression construct may then be isolated and introduced into a packaging cell transduced with a retroviral plasmid vector containing RNA encoding a VDCC γ-8 polypeptide such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

### Formulation and Administration

Peptides, such as the soluble form of VDCC γ-8 ion channel polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The disclosure further relates to pharmaceutical packs and kits comprising one or more containers filled with one or more of the ingredients of the aforementioned compositions.

VDCC γ-8 polypeptides and other compounds may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localize, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

### Pharmaceutical Compositions

A pharmaceutical composition comprising a therapeutically effective amount of the VDCC γ-8 polypeptide, polynucleotide, peptide, vector or antibody thereof and optionally a pharmaceutically acceptable carrier, diluent or excipients (including combinations thereof) is provided.

The pharmaceutical composition may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition described here may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Vaccines

Another embodiment relates to a method for inducing an immunological response in a mammal which comprises inoculating the mammal with the VDCC γ-8 ion channel polypeptide, or a fragment thereof, adequate to produce antibody and/or T cell immune response to protect said animal from VDCC γ-8-associated diseases.

Yet another embodiment relates to a method of inducing immunological response in a mammal which comprises delivering a VDCC γ-8 polypeptide via a vector directing expression of VDCC γ-8 polynucleotide in vivo in order to induce such an immunological response to produce antibody to protect said animal from diseases.

A further embodiment relates to an immunological/vaccine formulation (composition) which, when introduced into a mammalian host, induces an immunological response in that mammal to a VDCC γ-8 polypeptide wherein the composition comprises a VDCC γ-8 polypeptide or VDCC γ-8 gene. The vaccine formulation may further comprise a suitable carrier.

Since the VDCC γ-8 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Vaccines may be prepared from one or more VDCC γ-8 polypeptides or peptides.

The preparation of vaccines which contain an immunogenic polypeptide(s) or peptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al2O3 basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The VDCC γ-8 polypeptides may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and vaccine compositions described here may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (MV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

The term “co-administered” means that the site and time of administration of each of for example, the VDCC γ-8 polypeptide and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The VDCC γ-8 polypeptide, polynucleotide, peptide, nucleotide, antibody thereof and optionally an adjuvant may be administered separately or co-administered to the host subject as a single dose or in multiple doses.

The vaccine composition and pharmaceutical compositions may be administered by a number of different routes such as injection (which includes parenteral, subcutaneous and intramuscular injection) intranasal, mucosal, oral, intra-vaginal, urethral or ocular administration.

The vaccines and pharmaceutical compositions of described here may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

### EXAMPLES

### Example 1

### Transgenic VDCC γ-8 Knock-Out Mouse: Construction of VDCC γ-8 Gene Targeting Vector

The VDCC γ-8 gene was identified bio-informatically using homology searches of genome databases. A 40 kb murine genomic contig was assembled from various databases. This contig provided sufficient flanking sequence information to enable the design of homologous arms to clone into the targeting vector.

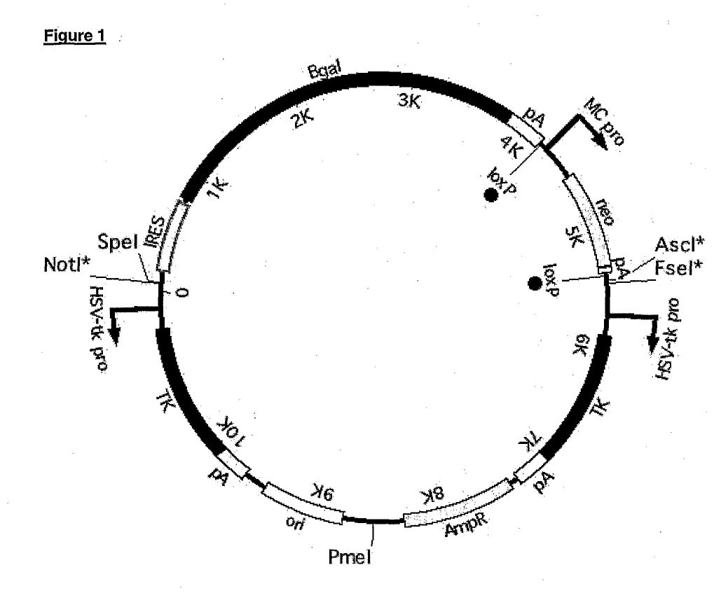

The murine VDCC γ-8 gene has 4 coding exons. The targeting strategy is designed to remove the majority of the translated part of the first coding exon. A 4.1 kb 5′ homologous arm and a 2.1 kb 3′ homologous arm flanking the region to be deleted are amplified by PCR and the fragments are cloned into the targeting vector. The 5′ end of each oligonucleotide primer used to amplify the arms is synthesised to contain a different recognition site for a rare-cutting restriction enzyme, compatible with the cloning sites of the vector polylinkers and absent from the arms themselves. In the case of VDCC γ-8, the primers are designed as listed in the primer table below, with 5′ arm cloning sites of NotI/SpeI and 3′arm cloning sites of AscI/FseI (the structure of the targeting vector used, including the relevant restriction sites, is shown in FIG. 1).

In addition to the arm primer pairs (5′armF/5′armR) and (3′armF/3′armR), further primers specific to the VDCC γ-8 locus are designed for the following purposes: 5′ and 3′ probe primer pairs (5′prF/5′prR and 3′prF/3′prR) to amplify two short 150-300 bp fragments of non-repetitive genomic DNA external to and extending beyond each arm, to allow Southern analysis of the targeted locus, in isolated putative targeted clones; a mouse genotyping primer pair (hetF and hetR) which allows differentiation between wild-type, heterozygote and homozygous mice, when used in a multiplex PCR with a vector specific primer, in this case, asc350; and lastly, a target screening primer (3′scr) which anneals downstream of the end of the 3′ arm region, and which produces a target event specific 2.2 kb amplimer when paired with a primer specific to the 3′ end of the vector (TK5IBLMNL), in this case asc146. This amplimer can only be derived from template DNA from cells where the desired genomic alteration has occurred and allows the identification of correctly targeted cells from the background of clones containing randomly integrated copies of the vector. The location of these primers and the genomic structure of the regions of the VDCC γ-8 locus used in the targeting strategy is shown in FIG. 7 (SEQ ID NO: 21).

TABLE 1
VDCC γ-8 Primer Sequences

| | |
|---|---|
| musPiom 5′prF (SEQ ID NO.8) | CGTCAGGGTTAAGCAATGGGGAAGCAG |
| musPiom 5′prR (SEQ ID NO.9) | CGCCACCGCCTCTTCCTGTTGTCATAG |
| musPiom 5′armF Not (SEQ ID NO.10) | aaagcggccGCCATGCTGAAGAAATGGAGGCTAGTG |
| musPiom 5′armR Spe (SEQ ID NO.11) | aaaactAGTAGTCAGTGCTGATGGCGATGGTC |
| musPiom 3′armF Asc (SEQ ID NO.12) | tttggcgcgCCTCCAGCAAATCGACTTCTAGTGTTC |
| musPiom 3′armR Fse (SEQ ID NO.13) | tttggccggccTGTTTTACCCCTTGTTGCTCTTGAATC |
| musPiom 3′scr (SEQ ID NO.14) | CCATTACTCTTTTCCCCCTCCTTCCAG |
| musPiom 3′prF (SEQ ID NO.15) | ACCCCAGGCACTGATGAAGAATTGAAG |
| musPiom 3′prR (SEQ ID NO.16) | CCCCCTGTCACGGGTATTAI1TCTTAC |
| musPiom hetF (SEQ ID NO.17) | GCTCTCATCTGCAACACCACCAAGCTC |
| musPiom hetR (SEQ ID NO.18) | GACTCAGAACACTAGMGTCGATTTGC |
| asc350 (SEQ ID NO.19) | GTCGTGACCCATGGCGATGCCTGCTTG |
| asc146 (SEQ ID NO.20) | CGCATCGCCTTCTATCGCCTTCTTGAC |

The position of the homology arms is chosen to functionally disrupt the VDCC γ-8 gene. A targeting vector is prepared where the VDCC γ-8 region to be deleted is replaced with non-homologous sequences composed of an endogenous gene expression reporter (a frame independent lacZ gene) upstream of a selection cassette composed of a promoted neomycin phosphotransferase (neo) gene arranged in the same orientation as the VDCC γ-8 gene.

Once the 5′ and 3′ homology arms have been cloned into the targeting vector TK5IBLMNL (see FIG. 1), a large highly pure DNA preparation is made using standard molecular biology techniques. 20 ug of the freshly prepared endotoxin-free DNA is restricted with another rare-cutting restriction enzyme PmeI, present at a unique site in the vector backbone between the ampicillin resistance gene and the bacterial origin of replication. The linearized DNA is then precipitated and resuspended in 100 μl of Phosphate Buffered Saline, ready for electroporation.

24 hours following electroporation the transfected cells are cultured for 9 days in medium containing 200 μg/ml neomycin. Clones are picked into 96 well plates, replicated and expanded before being screened by PCR (using primers 3′scr and asc146, as described above) to identify clones in which homologous recombination has occurred between the endogenous VDCC γ-8 gene and the targeting construct. Positive clones can be identified at a rate of 1 to 5%. These clones are expanded to allow replicas to be frozen and sufficient high quality DNA to be prepared for Southern blot confirmation of the targeting event using the external 5′ and 3′ probes prepared as described above, all using standard procedures (Russ et al, Nature 2000 Mar. 2; 404 (6773):95-99). When Southern blots of DNA digested with diagnostic restriction enzymes are hybridized with an external probe, homologously targeted ES cell clones are verified by the presence of a mutant band as well an unaltered wild-type band. For instance, using the 5′ probe, EcoRI digested genomic DNA will give a 9.3 kb wild-type band and a 6.7 kb targeted band; and with the 3′ probe, SspI cut DNA will give a 15 kb wild-type band and a 9.5 kb targeted band.

### Example 2

### Transgenic VDCC γ-8 Knock-Out Mouse: Generation of VDCC γ-8 Ion Channel Deficient Mice

C57BL/6 female and male mice are mated and blastocysts are isolated at 3.5 days of gestation. 10-12 cells from a chosen clone are injected per blastocyst and 7-8 blastocysts are implanted in the uterus of a pseudopregnant F1 female. A litter of chimeric pups are born containing several high level (up to 100%) agouti males (the agouti coat colour indicates the contribution of cells descended from the targeted clone). These male chimeras are mated with female MF1 and 129 mice, and germline transmission is determined by the agouti coat colour and by PCR genotyping respectively.

PCR Genotyping is carried out on lysed tail clips, using the primers hetF and hetR with a third, vector specific primer (asc350). This multiplex PCR allows amplification from the wild-type locus (if present) from primers hetF and hetR giving a 216 bp band. The site for hetF is deleted in the knockout mice, so this amplification will fail from a targeted allele. However, the asc350 primer will amplify a 384 bp band from the targeted locus, in combination with the hetR primer which anneals to a region just inside the 3′ arm. Therefore, this multiplex PCR reveals the genotype of the litters as follows: wild-type samples exhibit a single 216 bp band; heterozygous DNA samples yield two bands at 216 bp and 384 bp; and the homozygous samples will show only the target specific 384 bp band.

### Example 3

### Biological Data: Gene Expression Patterns

1) RT-PCR

Using RT-PCR, expression of the gene is shown in the hippocampus (+++), cerebellum, cortex, striatum, mid brain, pons (++), hypothalamus, thalamus, forebrain, spinal cord, pituitary, and trigeminal ganglion (+)

2) List of Lac Z Stained Structures

LacZ Staining

The X gal staining of dissected tissues is performed in the following manner.

Representative tissue slices are made of large organs. Whole small organs and tubes are sliced open, so fixative and stain will penetrate. Tissues are rinsed thoroughly in PBS (phosphate buffered saline) to remove blood or gut contents. Tissues are placed in fixative (PBS containing 2% formaldehyde, 0.2% glutaraldehyde, 0.02% NP40, 1 mM MgCl2, Sodium deoxycholate 0.23 mM) for 30-45 minutes. Following three 5 minute washes in PBS, tissues are placed in Xgal staining solution (4 mM K Ferrocyanide, 4 mM K Ferricyanide, 2 mM MgCl2, 1 mg/mIX-gal in PBS) for 18 hours at 30 C. Tissues are PBS washed 3 times, postfixed for 24 hours in 4% formaldehyde, PBS washed again before storage in 70% ethanol.

Using LacZ staining, VDCC γ-8 is found to be expressed in the hippocampus, cortex, amygdala, olfactory cortex, forebrain and thalamus.

### Example 4

### Biological Data: Behaviour: Rotarod

All animals are housed with free access to food and water under a light-dark cycle of 12 h light/12 h darkness with lights on at 7 am. Animals are tested at set times to avoid circadian effects. The test involves three trials on the accelerating rotarod, which accelerates at a constant rate from 4 to 40 rpm over the course of 5 min, then remains at 40 rpm until either the cut-off time (10 minutes), or when the mice fall off the rotardo. The time at which animals fall from the rotarod is recorded.

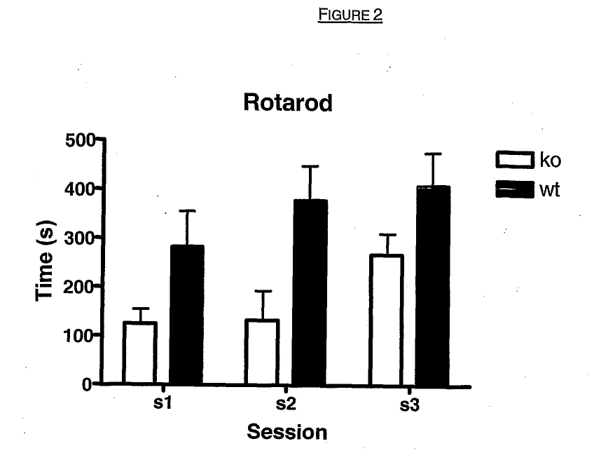

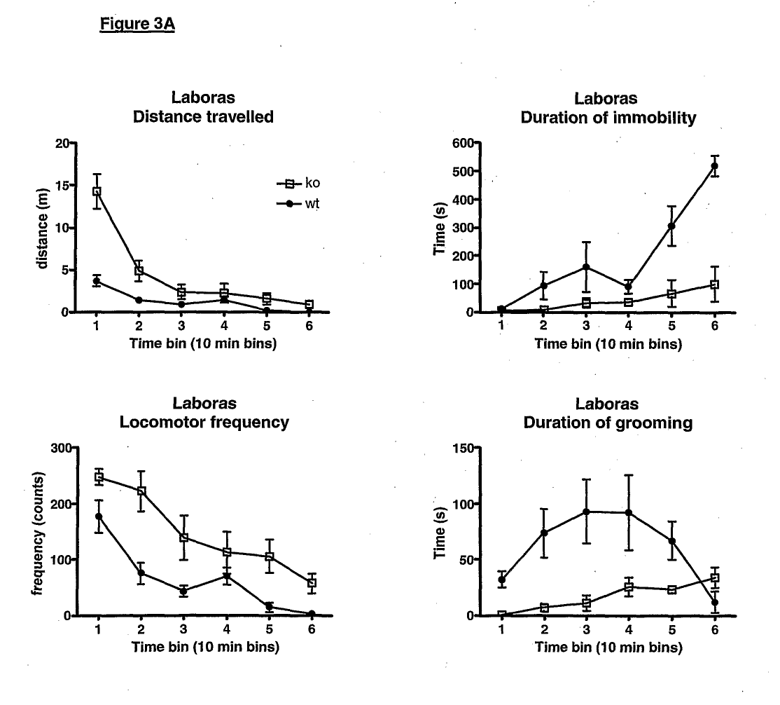

Knockout mice showed a reduced performance on the rotarod compared to wildtype mice, in all three sessions (FIG. 2).

### Example 5

### Biological Data: Behaviour: Laboras

The laboras (Laboratory Animal Behaviour Observation Registration and Analysis System) system is an automated behaviour apparatus commercialised by Metris BV (Netherlands). Laboras automatically records and quantifies behaviours using vibrations patterns. All animals are housed with free access to food and water under a light-dark cycle of 12 h light/12 h darkness with lights on at 7 am. Animals are tested at set times to avoid circadian effects. Mice are placed in this apparatus for an initial period of 1 hour with free access to food and water. This is followed up by a 15 hour overnight session with free access to food and water.

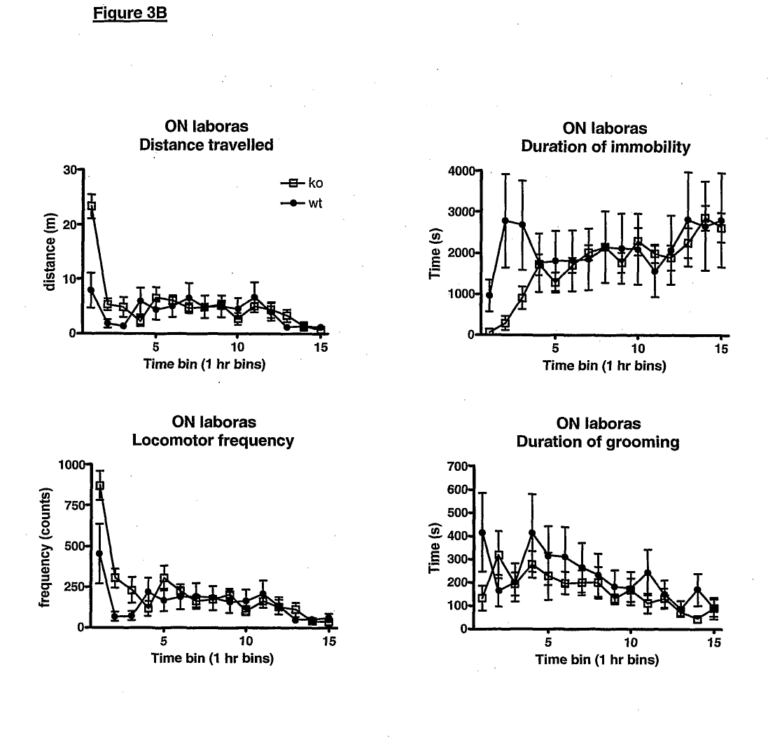

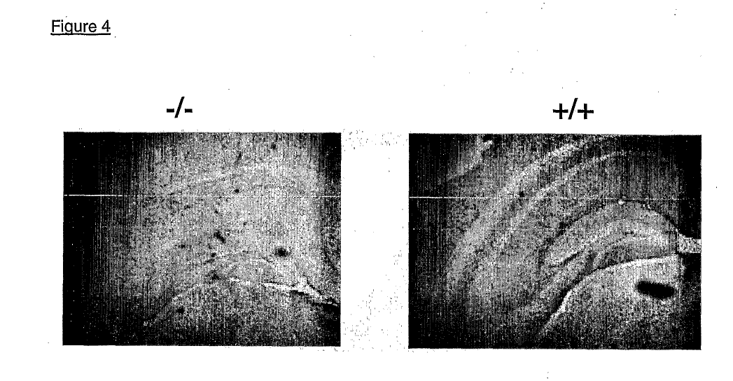

In the 1 hour study, VDCC γ-8 knockout mice are hyperactive (locomotor frequency/distance traveled/grooming) across the session (FIG. 3a).

In the 15 hour overnight study, VDCC γ-8 knockout mice are hyperactive for the first three hours of the session, after which time the level activity decreased as the animals habituated to the apparatus to match the activity levels of the wildtype mice (FIG. 3b).

### Example 5

### Biological Data: Immunohistochemistry: GluR2 Immunohistochemistry

Using standard immunohistological methods for GluR antibodies (e.g. Mead and Stephens (2003) J. Neurosci. 23: 1041-1048), immunolabelling of the GluR2 AMPA receptor subunit was completed in sections of the brain containing the hippocampus using Sant Cruz GluR-2 (N-19; sc-7611) antibody. Immunostaining for GluR2 was very low in the hippocampus of VDCC γ-8 knockout mice compared to wildtype mice (FIG. 4).

### Example 6

### Biological Data: Behaviour: Social Interaction

All animals are housed with free access to food and water under a light-dark cycle of 12 h light/12 h darkness with lights on at 7 am. Animals are tested at set times to avoid circadian effects.

Animals (5 KO mice of each sex with corresponding age matched wild type controls) are placed in a three chamber apparatus, consisting of a central chamber and two ‘side’ chambers each containing a 9 cm diameter cylinder. Each test mouse is initially placed in the apparatus for 5 minutes and the activity recorded using tracking software. The test mouse is then removed, an intruder mouse of the same sex placed on of the cylinders and the test mouse then placed back into the central chamber. The activity is then recorded for a further five minutes. The time each test mouse spends in the chamber containing the intruder mouse, compared to the empty chamber is recorded and used as a measure of social interaction. The data is calculated as the time or percentage time spent in each chamber or as the approach avoidance score, where a positive score indicates increasing social interaction, a score of zero indicates no social interaction and a negative score indicates increasing social avoidance.

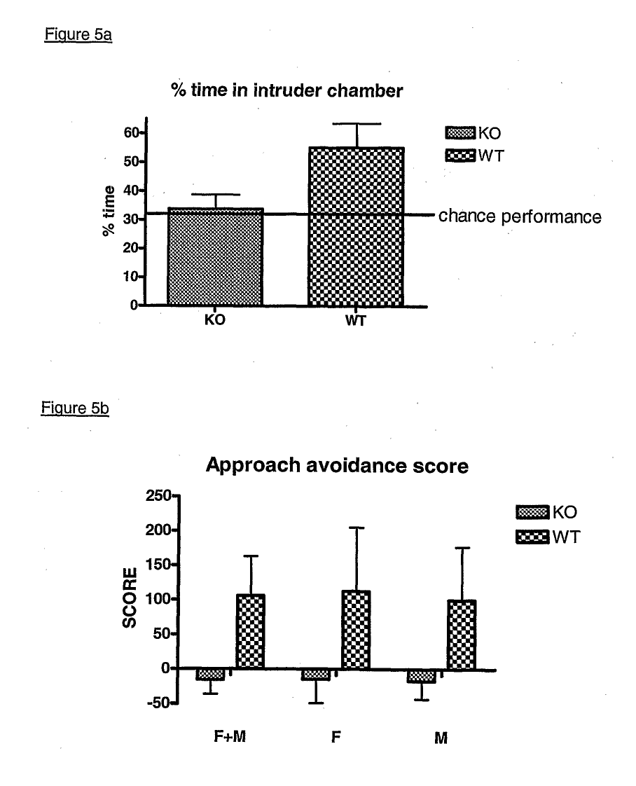

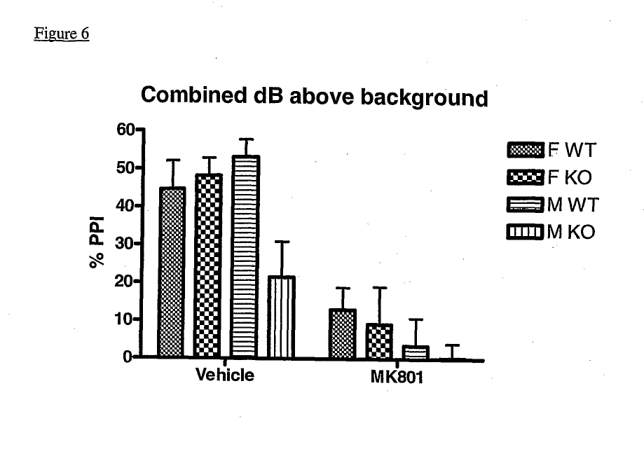

VDCC γ-8 knockout mice showed a clear lack of social interaction in this test compared to aged matched wild type control when measured as percentage time in the intruder chamber or as approach avoidance score (FIGS. 5a& 5b).

This data suggests utility in schizophrenia.

### Example 7

### Biological Data: Behaviour: Pre Pulse Inhibition,

Pre pulse inhibition (PPI) is an observed phenomenon whereby the startle response evoked by a loud (115 dB) stimulus is inhibited by a brief much quieter sound stimulus (3-10 dB above background) presented 100 ms before the startle stimulus. This so-called pre pulse inhibition is disrupted in schizophrenic patients and may reflect a deficit in psychomotor gating.

PPI can be evoked in rodents and disrupted by psychogenic and psychotomimetic drugs such as amphetamine, apomorphine, phencyclidine and MK801. This disruption can be reversed by antipsychotic drugs such as haloperidol, clozapine and olanzapine. (Bakshi V P, Swerdlow N R and Geyer M A. Clozapine antagonizes phencyclidine-induced deficits in sensorimotor gating of the startle response. J. Pharmacol. Exp. Ther. 1994; 271:787-794). Therefore an animal model of PPI exists with both construct and predictive validity Any effect of a drug or a mutation on PPI is a strong indication of potential utility in the therapy of schizophrenia. (McGhie A and Chapman J. Disorders of attention and perception in early schizophrenia. Br. J. Med. Psychol. 1961; 34:103-116. Braff D L, Geyer M A and Swerdlow N R. Human studies of prepulse inhibition of startle: normal subjects, patient groups, and pharmacological studies. Psychopharmacology. 2001; 156:234-258) Animals are housed with free access to food and water under a light-dark cycle of 12 h light/12 h darkness with lights on at 7 am and are tested at set times to avoid circadian effects. Mice (6 KO mice of each sex with corresponding age matched wild type controls) are placed in a startle chamber (San Diego Instruments, San Diego Calif., USA). Each system consisted of a sound attenuated chamber ventilated with a small fan and illuminated by a light bulb mounted in the ceiling. The chamber contained a transparent acrylic cylinder (where mouse to be put in) mounted to a frame. The cylinder and the frame were elevated about a Plexiglas base by four screws stationed under each corner of the frame. Acoustic stimuli were delivered through a small speaker mounted in the enclosure ceiling. Startle responses were transduced by the piezoelectric accelerometer mounted beneath the frame. Output signals were digitized, rectified and recorded as consecutive 1 millisecond (ms) readings on a PC with San Diego Instruments Startle Reflex software. The startle stimulus was a 115 dB pulse of broad spectrum noise 50 ms in length. The pre-pulse stimulus was 3, 7 and 10 dB above background and presented 100 ms before the startle stimulus. Stimuli were presented in a pseudo randomised fashion. In the male KO mice PPI was significantly disrupted compared to age matched wild type controls (paired t test p=0.01), see FIG. 6.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in molecular biology or related fields are intended to be within the scope of the claims. Furthermore, various combinations of the features of the following dependent claims can be made with the features of the independent claims without departing from the scope of the present invention.

1. A method of identifying an agent suitable for therapy of a mental illness, comprising the step of determining whether a candidate agent affects the activity of voltage-dependant calcium-channel gamma-8.

2. The method according to claim 1, wherein the agent is an antagonist that inhibits the activity of voltage-dependent calcium channel gamma-8.

3. The method according to claim 1, wherein the agent is an agonist that enhances the activity of voltage-dependent calcium channel gamma-8.

4. The method according to claim 1, wherein the voltage-dependant calcium channel gamma-8 comprises, or encodes, a polypeptide identified herein as SEQ ID No. 3, SEQ ID No. 5 or SEQ ID No 7, or a sequence with at least 70% sequence identity thereto.

5. The method according to claim 1, wherein the voltage-dependent calcium channel gamma-8 consists of, or encodes, a polypeptide identified herein as SEQ ID No 3, SEQ ID No 5, SEQ ID No 7, or a sequence with at least 70% identity thereto.

6. The method according to claim 1, wherein the mental illness involves psychosis.

7. The method according to claim 1, wherein the mental illness is schizophrenia, attention-deficit/hyperactivity disorder, bipolar disorder or major depression.

8. The method according to claim 1, wherein the mental illness is epilepsy.

9. The method according to claim 1, wherein voltage-dependent calcium-channel gamma-8 polypeptide is contacted with the candidate agent to determine whether the candidate affects the activity of the voltage-dependent calcium-channel gamma-8 polypeptide.

10. The method according to claim 1, wherein the candidate agent is contacted with a cell expressing a voltage-dependent calcium-channel gamma-8 polypeptide.

11. The method according to claim 1, wherein the candidate agent is administered to a non-human animal expressing a voltage-dependent calcium-channel gamma-8 polypeptide and determining whether the animal exhibits altered behaviour.

12. The method according to claim 11, wherein the non-human animal expresses functional voltage-dependent calcium-channel gamma-8 polypeptide.

13. The method according to claim 11, wherein the non-human animal is wild-type.

14. The method according to claim 11, wherein the non-human animal is a rodent.

15. A non-human transgenic animal having a functionality-disrupted endogenous voltage-dependent calcium-channel gamma-8 gene.

16. The non-human transgenic animal according to claim 15, wherein the animal has a null mutation.

17. The non-human transgenic animal according to claim 15, wherein the animal is −/− for the voltage-dependent calcium-channel gamma-8 gene.

18. (canceled)

19. Use of a non-human transgenic animal according to claim 15 as a model for mental illness.

20. A method of determining the presence or susceptibility of mental illness in an individual, comprising the steps of (i) determining the expression level of voltage-dependent calcium-channel gamma-8 gene in a sample isolated from the patient, and (ii) determining, compared to a control, whether the individual from which the sample was isolated has or is susceptible to mental illness.

21. Use of 1) an exogenous voltage-dependent calcium-channel gamma-8 polynucleotide; 2) an agent that affects the activity of a voltage-dependent calcium-channel gamma-8 polypeptide; or 3) an agent that increases or decreases the transcription or expression of a voltage-dependent calcium-channel gamma-8 polynucleotide in the manufacture of a medicament for the prevention or treatment of mental illness.

22-23. (canceled)

24. The method according to claim 20, wherein the mental illness involves psychosis; or is schizophrenia, attention-deficit/hyperactivity disorder, bipolar disorder or major depression; or the mental illness is epilepsy.

25. The method, according to claim 1, wherein said method utilizes a non-human transgenic animal having a functionality-disrupted endogenous voltage-dependent calcium-channel gamma-8 gene.


SEQUENCE LISTING (21 sequences)

SEQ ID NO: 1 - DNA, 1417 bp, Homo sapiens
ccccgcttctgcctgcgctgtgaacccccccccagccgccggcacggccccgcccccgctgccccggtggtggcccacggccccccggctgcccgtggtcaaactggagtcgctgaagcgctggaacgaagagcggggcctctggtgcgagaagggggtgcaggtgctgctgacgacggtgggcgccttcgccgccttcggcctcatgaccatcgccatcagcactgactactggctctacacgcgcgccctcatctgcaacaccaccaacctcacggccggcggcgacgacgggaccccccaccgcgggggcggcggcgcctcggagaagaaggaccccggcggcctcacgcactcgggcctctggaggatctgctgcctggaagggttgaaaagaggcgtctgcgtgaagatcaatcatttcccggaggacacggactacgaccacgacagcgcggagtatctactccgagttgtccgggcctccagcatcttccccatccttagcgccatcctgctgctgctcgggggtgtgtgcgtggcggcctcccgcgtctacaagtccaagaggaacatcattctgggcgcagggatcctgttcgtggcagcaggcctgagcaacatcatcggcgtgatcgtgtacatctccgccaacgcgggcgagccgggcccgaagcgggacgaggagaagaaaaaccactactcgtacggctggtccttctacttcggcgggctgtcgttcatcctggccgaggtgataggcgtgctggccgtcaacatctacatcgagcgcagccgcgaggcgcactgccagtctcgctcggacctgctcaaggccggcgggggcgcgggcggcagtggcgggagcggcccctcggccatcctccgtctgcccagttaccgcttccgctaccgccgccgctcccgctctagctcccgctccagcgagccgtcgccgtcgcgggacgcgtctcccggcggccccgggggcccgggctttgcctccacggacatctccatgtacacgctcagccgcgacccctccaagggcagcgtggccgcggggctggcgggggccggcggcggcggcggcggcgccgtgggggcgttcggcggcgcggccgggggcgccgggggcggcggcggaggcggcggcggggcgggtgccgagcgggaccgcgggggggcgtccggcttcctcacgctgcacaacgccttccccaaggaggcgggcggcggcgtcacggtcacggtcaccgggccgcccgccccgcccgcgcccgcgccacccgcgccctctgcgcccgcccccgggaccctggccaaggaggccgccgcctccaacaccaacacgctcaacaggaaaaccacgcctgtgtaggggcgcggcgggggagccgaggggcgtgtccggggc

SEQ ID NO: 2 - DNA, 1278 bp, Homo sapiens
ctggagtcgctgaagcgctggaacgaagagcggggcctctggtgcgagaagggggtgcaggtgctgctgacgacggtgggcgccttcgccgccttcggcctcatgaccatcgccatcagcactgactactggctctacacgcgcgccctcatctgcaacaccaccaacctcacggccggcggcgacgacgggaccccccaccgcgggggcggcggcgcctcggagaagaaggaccccggcggcctcacgcactcgggcctctggaggatctgctgcctggaagggttgaaaagaggcgtctgcgtgaagatcaatcatttcccggaggacacggactacgaccacgacagcgcggagtatctactccgagttgtccgggcctccagcatcttccccatccttagcgccatcctgctgctgctcgggggtgtgtgcgtggcggcctcccgcgtctacaagtccaagaggaacatcattctgggcgcagggatcctgttcgtggcagcaggcctgagcaacatcatcggcgtgatcgtgtacatctccgccaacgcgggcgagccgggcccgaagcgggacgaggagaagaaaaaccactactcgtacggctggtccttctacttcggcgggctgtcgttcatcctggccgaggtgataggcgtgctggccgtcaacatctacatcgagcgcagccgcgaggcgcactgccagtctcgctcggacctgctcaaggccggcgggggcgcgggcggcagtggcgggagcggcccctcggccatcctccgtctgcccagttaccgcttccgctaccgccgccgctcccgctctagctcccgctccagcgagccgtcgccgtcgcgggacgcgtctcccggcggccccgggggcccgggctttgcctccacggacatctccatgtacacgctcagccgcgacccctccaagggcagcgtggccgcggggctggcgggggccggcggcggcggcggcggcgccgtgggggcgttcggcggcgcggccgggggcgccgggggcggcggcggaggcggcggcggggcgggtgccgagcgggaccgcgggggggcgtccggcttcctcacgctgcacaacgccttccccaaggaggcgggcggcggcgtcacggtcacggtcaccgggccgcccgccccgcccgcgcccgcgccacccgcgccctctgcgcccgcccccgggaccctggccaaggaggccgccgcctccaacaccaacacgctcaacaggaaaaccacgcctgtgtag

SEQ ID NO: 3 - protein, 425 aa, Homo sapiens
MESLKRWNEERGLWCEKGVQVLLTTVGAFAAFGLMTIAISTDYWLYTRALICNTTNLTAGGDDGTPHRGGGGASEKKDPGGLTHSGLWRICCLEGLKRGVCVKINHFPEDTDYDHDSAEYLLRVVRASSIFPILSAILLLLGGVCVAASRVYKSKRNIILGAGILFVAAGLSNIIGVIVYISANAGEPGPKRDEEKKNHYSYGWSFYFGGLSFILAEVIGVLAVNIYIERSREAHCQSRSDLLKAGGGAGGSGGSGPSAILRLPSYRFRYRRRSRSSSRSSEPSPSRDASPGGPGGPGFASTDISMYTLSRDPSKGSVAAGLAGAGGGGGGAVGAFGGAAGGAGGGGGGGGGAGAERDRGGASGFLTLHNAFPKEAGGGVTVTVTGPPAPPAPAPPAPSAPAPGTLAKEAAASNTNTLNRKTTPV

SEQ ID NO: 4 - DNA, 1380 bp, Mus musculus
tgaggccccccagcttctgcctgtgctgtgatccccccagccgccggcacggccccgcctccgctgccccggtggtggcccacggccccccggttgccagtggtgaaactggagtcattgaaacgctggaatgaagagaggggtttgtggtgtgaaaagggcgttcaggtactactgaccaccatcggcgccttctcggcttttggcctcatgaccatcgccatcagcactgactactggctctacacaagagctctcatctgcaacaccaccaacctcacagcaggtgatgacggaccaccccatcgtgggggcagtggctcctccgagaagaaggaccctgggggcctcacacattcaggcctctggcggatatgctgcctggaagggttgaaaagaggtgtctgcgtgaagatcaaccacttcccggaggacacggactacgaccacgacagcgcggagtacctgctccgagtagtccgggcttccagcatctttcctatcctgagcgccatcctgctgctgctcgggggcgtgtgcgtagctgcctcccgcgtctacaagtccaaaaggaacatcatcctgggcgcagggatcctgttcgtggcagcaggcttgagcaacatcatcggggtgattgtgtacatatcggccaacgccggcgagcctggccccaagagggacgaggagaagaaaaaccactactcgtacggctggtccttctacttcggcgggctgtccttcatcctggctgaggtgatcggagtactggccgtcaacatctacatcgagcgcagccgcgaggcacactgccaatcacgctcggacctgctcaaggccggcggcggcgcgggcggcagtggcgggagcggcccctcggccatcctccgtctgcccagttaccgcttccgctaccgccgccgctcccgctccagctcccgaggctccagcgaggcgtcgccatcccgggatgcgtctcccggcggccccgggggcccgggcttcgcctccacggacatctccatgtacacgctcagccgcgacccgtccaagggcagcgtggctgcggggctggcgagcgccggtggcggcggcagcggtgccggcgtgggtgcctacggcggggcggccggggcggcggggggcggcggggcgggctcggagcgggaccgcgggagctcggcgggttttctcacgctgcacaacgccttccccaaggaagcggcgtccggcgtcacggtcacagtcaccggaccgcccgctgcacccgcgcccgcgcccgcgccgcccgctcctgcagcgcccgcgcccgggaccctgtccaaagaggccgcggcgtccaacaccaacacgctcaacaggaaaaccacgcccgtgtag

SEQ ID NO: 5 - protein, 423 aa, Mus musculus
MESLKRWNEERGLWCEKGVQVLLTTIGAFSAFGLMTIAISTDYWLYTRALICNTTNLTAGDDGPPHRGGSGSSEKKDPGGLTHSGLWRICCLEGLKRGVCVKINHFPEDTDYDHDSAEYLLRVVRASSIFPILSAILLLLGGVCVAASRVYKSKRNIILGAGILFVAAGLSNIIGVIVYISANAGEPGPKRDEEKKNHYSYGWSFYFGGLSFILAEVIGVLAVNIYIERSREAHCQSRSDLLKAGGGAGGSGGSGPSAILRLPSYRFRYRRRSRSSSRGSSEASPSRDASPGGPGGPGFASTDISMYTLSRDPSKGSVAAGLASAGGGGSGAGVGAYGGAAGAAGGGGAGSERDRGSSAGFLTLHNAFPKEAASGVTVTVTGPPAAPAPAPAPPAPAAPAPGTLSKEAAASNTNTLNRKTTPV

SEQ ID NO: 6 - DNA, 1176 bp, Homo sapiens
atgaccatcgccatcagcactgactactggctctacacgcgcgccctcatctgcaacaccaccaacctcacggccggcggcgacgacgggaccccccaccgcgggggcggcggcgcctcggagaagaaggaccccggcggcctcacgcactcgggcctctggaggatctgctgcctggaagggttgaaaagaggcgtctgcgtgaagatcaatcatttcccggaggacacggactacgaccacgacagcgcggagtatctactccgagttgtccgggcctccagcatcttccccatccttagcgccatcctgctgctgctcgggggtgtgtgcgtggcggcctcccgcgtctacaagtccaagaggaacatcattctgggcgcagggatcctgttcgtggcagcaggcctgagcaacatcatcggcgtgatcgtgtacatctccgccaacgcgggcgagccgggcccgaagcgggacgaggagaagaaaaaccactactcgtacggctggtccttctacttcggcgggctgtcgttcatcctggccgaggtgataggcgtgctggccgtcaacatctacatcgagcgcagccgcgaggcgcactgccagtctcgctcggacctgctcaaggccggcgggggcgcgggcggcagtggcgggagcggcccctcggccatcctccgtctgcccagttaccgcttccgctaccgccgccgctcccgctctagctcccgctccagcgagccgtcgccgtcgcgggacgcgtctcccggcggccccgggggcccgggctttgcctccacggacatctccatgtacacgctcagccgcgacccctccaagggcagcgtggccgcggggctggcgggggccggcggcggcggcggcggcgccgtgggggcgttcggcggcgcggccgggggcgccgggggcggcggcggaggcggcggcggggcgggtgccgagcgggaccgcgggggggcgtccggcttcctcacgctgcacaacgccttccccaaggaggcgggcggcggcgtcacggtcacggtcaccgggccgcccgccccgcccgcgcccgcgccacccgcgccctctgcgcccgcccccgggaccctggccaaggaggccgccgcctccaacaccaacacgctcaacaggaaaaccacgcctgtgtag

SEQ ID NO: 7 - protein, 391 aa, Homo sapiens
MTIAISTDYWLYTRALICNTTNLTAGGDDGTPHRGGGGASEKKDPGGLTHSGLWRICCLEGLKRGVCVKINHFPEDTDYDHDSAEYLLRVVRASSIFPILSAILLLLGGVCVAASRVYKSKRNIILGAGILFVAAGLSNIIGVIVYISANAGEPGPKRDEEKKNHYSYGWSFYFGGLSFILAEVIGVLAVNIYIERSREAHCQSRSDLLKAGGGAGGSGGSGPSAILRLPSYRFRYRRRSRSSSRSSEPSPSRDASPGGPGGPGFASTDISMYTLSRDPSKGSVAAGLAGAGGGGGGAVGAFGGAAGGAGGGGGGGGGAGAERDRGGASGFLTLHNAFPKEAGGGVTVTVTGPPAPPAPAPPAPSAPAPGTLAKEAAASNTNTLNRKTTPV

SEQ ID NO: 8 - DNA, 27 bp, Artificial
  Synthetic Oligonucleotide Primer
cgtcagggttaagcaatggggaagcag

SEQ ID NO: 9 - DNA, 27 bp, Artificial
  Synthetic Oligonucleotide Primer
cgccaccgcctcttcctgttgtcatag

SEQ ID NO: 10 - DNA, 36 bp, Artificial
  Synthetic Oligonucleotide Primer
aaagcggccgccatgctgaagaaatggaggctagtg

SEQ ID NO: 11 - DNA, 32 bp, Artificial
  Synthetic Oligonucleotide Primer
aaaactagtagtcagtgctgatggcgatggtc

SEQ ID NO: 12 - DNA, 36 bp, Artificial
  Synthetic Oligonucleotide Primer
tttggcgcgcctccagcaaatcgacttctagtgttc

SEQ ID NO: 13 - DNA, 38 bp, Artificial
  Synthetic Oligonucleotide Primer
tttggccggcctgttttaccccttgttgctcttgaatc

SEQ ID NO: 14 - DNA, 27 bp, Artificial
  Synthetic Oligonucleotide Primer
ccattactcttttccccctccttccag

SEQ ID NO: 15 - DNA, 27 bp, Artificial
  Synthetic Oligonucleotide Primer
accccaggcactgatgaagaattgaag

SEQ ID NO: 16 - DNA, 27 bp, Artificial
  Synthetic Oligonucleotide Primer
ccccctgtcacgggtattatttcttac

SEQ ID NO: 17 - DNA, 27 bp, Artificial
  Synthetic Oligonucleotide Primer
gctctcatctgcaacaccaccaacctc

SEQ ID NO: 18 - DNA, 27 bp, Artificial
  Synthetic Oligonucleotide Primer
gactcagaacactagaagtcgatttgc

SEQ ID NO: 19 - DNA, 27 bp, Artificial
  Synthetic Oligonucleotide Primer
gtcgtgacccatggcgatgcctgcttg

SEQ ID NO: 20 - DNA, 27 bp, Artificial
  Synthetic Oligonucleotide Primer
cgcatcgccttctatcgccttcttgac

SEQ ID NO: 21 - DNA, 8060 bp, Mus musculus
cgtcagggttaagcaatggggaagcagagggtaccaagaaaaagaaaattgttggtaaaactcaaaatgcttgctgtgcttgtaaatgaatgatggttcatttggaagcaatctttggccccgctacttgagaaaagaggagaaaagacctgggaagctgagcttttccttgattttttttgggggggggggaagaggtggggagggcattcctggggtctttttaaaaaggccctccccttatttccggccgccgctgccaaccttggctgggcgcctggatcctgttgctatgacaacaggaagaggcggtggcggcggcaagccatgctgaagaaatggaggctagtgggggtgggggtggtggctttgaaatggggttgggggtagggatcaaggggattggggtgcgggtgagagggagcagaggtgtcctctatctctgacttcctcagtgcacctgaaatggtcccgtttcccacaatcaacctctctctctctctctctctctctctctctctctctctctctctctctctctctctctactctctctctctcccttgccccatccccaatttcttcatcaccccttcctctatttctttccctcttcccatcctctcaatctctctcttcccatctgtctctcctcccttttctctcccttatctcttactcctgttcccttccgttgcccagaccccatccctcttcctctcaaccccccccccaccttcccccagcttccccctacccttctatttctccctcatctcttgctgttctctctttgcatctcttcctgcttttgtccccttctctctctggatatttctctctgtgtgtgtctcctcaacttccttctgctaccagtcactgtctcccccaaatccttccctcccccacctctgttctcagtctctggatctctctgtttttccgattctccttgtctctcttcatctccctccctctctggatctgtttctcttcttcctcagcttccttctgccactcagaccctgctccccgaccacttcccttccccccaatccctctcctctcggaggctcccccatccctcagcagctcccctccccctccctccctcctccctccttctctcttccagctccgcactttacccagcgacacgaaaaccaaattgtctcctcacctttttttttttttttttttttttttaaatcccgaccctcatatcctgaccgcccaagctccgagctacccccggctcccctccccctcactctacacccccattcttcatacgcctcctcatctccatcccgtctcaaccaggtcggacgcctgggtcccttgctgttgggggagggggaagcagcaagaattgggagcccagaggtgttttttgggggggtgctggcaacaggggctggacgtccctccccagtgcatcggagagttgacctatcgtcaactgaggtgagagatctttttattaatcggaggtgggtgggtgtagaattaaggttctgtggatccccctgattctgagaaaagctggtctctcagatgaaggactgtgatcccttcttctatccagaagctttagaactgggggggggtcccctattcctttctcctttgcagatggactcaatgaagagtctccaaagcaaaacagattgcctctcaaattgacaaaaacaaaaagctcttaaaaatctttattgatgaagaaagtccctcctggtcccttttccttcaattccaggttgtagggcccactaggcatgggaaagaggtacatctcaggaccagagctcacgtctgtggaagaaacaatttgttaagatagtggaggtagctgctgtggtactggtgaggggcaaaggcatgtccagaggcaagagagaaactcagtccgggtcaggggccagcctggaaaatggacagctggaatgagcccagaaaggggggggggcagagagagagagagagagagagagaatgagagagagaaaatgagagagagagaatgagagagagaaggcaggagcacacgtgtggggcactaggccccgttgccatggagacaccccccccatagacaacctttctctacatcccctcctgggagcagacccagtccagggccagctggtgggggcggtggagccagggagaggaaactgggaggccaaggtcaccctgtaccaggacccagggtttctggttgctgcctctcatcctcagagcttccagactttcctacagggggattggaaagaagtcaagggaagaggttagaacaggtaaaggaaacagagaggaagaggcactatggtgggattgtatagaagacagaaatgaatgcacagcaatgagttatcccgagagttgtagtttgtagctttctccctttctcccccccgccttttttttttttttttttgagatggtgggggctaagtttctgcacaggtgaaattgttatggggaagggctggggttgttccagagaagtctatggggcctgcagaggggattttcctacatgaaagaggtgctagaagctgtggtgggtggaagaagtgggcaaaaggaaggggattttctacgatgtttttaaaaacaaaagttgggagggcaggttctaatatgttgaagagagtatagcggcagagtcacctgagagcatctagaactgggaaattactgaagctgtggtgaggtgtctctgtggagggcaagagccagggcatgctgggcagaacaggagacggcgattacatttttggacgggttagatattaggacatttctgaatggaaggaaattggggtctggagggcgtaatcgctgtgagtggggaagctgtggtgctcacagagcaaatttttcttaatggagaagaaagcagtggaaaagatgagtttgtgtaagttgtagggagaagggaggccaggttccaaatgcttcgaagcaagacagagtacagagttttctttgagttgaaaccacagtcggctgctggggttggaagggagatggatgacagtacaggagggaccgagagtccaaatggtgcaggtggggggtatagtctcttaaacttaggggacttggaaatagtccagggtgctttctaaaaaccctaggtgctctgactatttctcagatccattaaattgggatttctcaggtaggagtccaggcatcagaatttttattgaatcctctggcacttcagagatgtgtccgaggtgcaaggctagttcctaaggcacggaggctacgagaggaagagaggcctattaggacaaaactgtggaagtgttaggaccagtgagcagacattgaactcctgtgaatagtgatggtcacttggaaaggttttttttttttaagttaaggggacagtgagggatttatgtttcctaagtagagaaaattataagaagaaatgcaggtgtcacagaaacttttggggagattggcatcctgctaagaaggagaaagaaaagaaaaggactttgtgagcattttctttttcctttacatgggctgtcaagtggcccagacttgcctccaacttattatgtagccaaggacgcccttgaactgatccttctgcctctacctactgagtgctgacattacaggcatacaccaccatgccccatttatactgtggctgggatcaaacccagggctttgctaggccaacacctaccaactacatcccctgacttagtctctgacagttgctgctcctaagttggagagatactagagaggcacagttggagacgtatgaatcggcttttcttgagcagtgaacctacaaggtgcaggagtatgtgtgtggagtacgatatgtgagagagttggtagtggggagcagggggctctgaggagtgaacagaggcaggggcatccttaggaggagatagcagtagggttggaattgagggctggcttcacaagaggacagaggtgatcacagaagaggaactcaggggactggattatgacctcttagtatatggctagtgagggataatctctcagtagaatttagcaggcaagcagttttgggtaaaaaaaaaatgccatctcagagaactaactccctctctcttttgcctccaccagctggagactatggcttcactctgaccttcgcctctgcagccacctgctcgctgcccccggccccctctggccccttgaggccccccagcttctgcctgtgctgtgatccccccagccgccggcacggccccgcctccgctgccccggtggtggcccacggccccccggttgccagtggtgaaactggagtcattgaaacgctggaatgaagagaggggtttgtggtgtgaaaagggcgttcaggtactactgaccaccatcggcgccttcgcggcttttggcctcatgaccatcgccatcagcactgactactggctctacacaagagctctcatctgcaacaccaccaacctcacagcaggtgatgacggaccaccccatcgtgggggcagtggctcctccgagaagaaggaccctgggggcctcacacattcaggcctctggcggatatgctgcctggaaggtagggtgcaggaaggccctggctggccacctccgctgaagcctgtgcctccagcaaatcgacttctagtgttctgagtctcatcccaaggtcttgaggggtgtgctttactgctggaggcgtgtgattccatgcacccctccatccctctgtccaatccctcttctgggattctcgttttatttttattttatactggacttgatgatgcgtgtgaatgttttgtcagcgtggtcaactgtgcactgcatgcgtgcctggtgccctttgaggtcagaagagcctctggaactggagttacagatggttgtgtgccaggtggaattaccatatgtgtatctgtatgggtctctgctttgcacacttgcatgtattcaagtacccgtggaagcctgaacagggcctctgacacacatggaggtagtaataaagtgttgttagctgcctgatatggtgctgggaactggactctggtcctgtggaagagcagggagtgttcttcagtactgagccatctctccaacccccttaaaaagtgtgtgtgttttttttaattcatagtatttatttatttagtgtgtgctcgttccagtatgcaaatggaaggcagacggcaactcgcaggagttgataatcttcttcaaccatgtgggtccctgggatggaaactcaggttgtcacatttggccacatgtgtgaccttcacccacgagccatctcacatgcttgtcttgctccttccccatgtcttccctctcctgtttggctctgtgggctcccagaattgaaacaaagctatgccttatgacctcctcgggtctccctcggtagctcctcttcttgtaggacttttcttctcatctgcaaccttcggcttctctcccatcaggatctctcccaagctcacagcctggtcttccttgttcccatttccaatggcatctccccagcctctcttagcaggctccagggacatttcctttcttgtttctgctccccctccaatgctccccctccaaccggtttcttttctttgtgatgcttcctggctcaaacccctctccttgctagtcccctcattccccaagacccctgctgtcttcctcttcagccccttctctagcctgctttccatgtcagtaattctttcatgcacatctcaggtgtgccaccatgcatttagaggtgttgggctgggaagcaagactctgggagccccactgtgtgaccaccacccatcctctctgagccttggcgcccttatcagtggctttaaatagtggaccagggacctcgaaatatcccccatagcaccaataggcttaagtttttttattccccagctttgctttttcattcgtcttcatttccattgcctctgactcctctgtcctgacaaaccagtcaccaaacttgaggaatgggagggctttgaagataagtttgtggagtgtttcattacagaaaaccaaagtcagtcgtcaatgtatcaaagccggggagaccgcttcctgaaggtctgactgctggctcctggccagccatcatggaagatctgaccctaaaggcccgggagttctgaactgacccctactgcccctgactgagtagtgtgaactttcctggatctgattatggagtcactcagatatcatagtcaccagtgctcactgtcatcccctgcccctcttctctgtcactccccaccgtatccctggaggctgtgtcactcacgtgcatactctggcctggccaggcacagggtacagagccaaatacgagagtttgcacttggaccctccatttatgtatccactcactcatttatccggatgctcaagtacccatcagtttttattggctttaaactgcgtagcaaggttctgaagcttttaactttatgcgtgtgtcagtaggggctgagtgtgtgtctagctcagtgtgagcgtagaggcctgaggagaacttgtgggtgtcatttcgctctttccaccatgtaagttcgtggacctgaatcaggttctcaggcttggaagcaagaacctctactcactgagccatcgctgtggcccctaggttctcatggctgattcaagagcaacaaggggtaaaacatgcagatctctgactttaagtatcttctgctctggaaggagggggaaaagagtaatggagataaacaaatagggaaaaagcagaaatggagagagggaagcatcaggcctgtgcatgtgagtcaatcaagaagtctgggaggaggggctggagagatggctcagtggttaagagcactgactgctcttctgaaggtcctgagttcaaatcccagcaatcacatggtggctcacaaccatccataacaagaagtctgggaggagccagatggtggtggaagatgccttaatcccagcagaggcaggtggatctctgagttcaaggccagcctggtctatagagtgagttccaggacagccagggctacacagagaagccctgtctcgaaaaccaaaaatgaaagaaagaaagacaagtggagggaggaattccacgtggcagttgactttggttggtgaccagaaagaagtgaggttgaagacacagggtgttgggctgtggatatagtccttgcaaaggtcctgaggtagcgtgaatctgtgtttggccaacagtgggagaccagtgtggctgggacacaatgagaggggagagatgtatggtcaaggagataatagacacttgatcccacaggttcacatatatggcatagaagattcagcttttgcttggagatgggagtccctgaaggttccggagcagagaagccctgtgagctagcttaagttctaccaggatggctcctgctgctgtgctcagaaatggctacggggagggtggaaagggagaatgtgggagccagtggtttgaatgcatagtatattccaaagttccccgtgggaaggcactgctgatggagcaattatggcatctgagagggagagcagggtgaggggtgtctcggaggtggggcttctgaacctccgccctggtgacattttgggactgagaattctgtgtggcgggggctgagctgtacatcgtaggacgttgatctgctcctctattctctgtctagtcccgcccacccccaacagttgtgacaaccaagatcgtaaaattgtctccatacattatttacattctcgggggggggggggggggagattaaatatcccctcaggtgagaaccccaggcactgatgaagaattgaagaagtgctggccagagcacttggcagtgggggatctgctcttgagatgaaagagcaagcagtttttatttaaattcctttattttctcttagttagctcactattaaggagaggtagcacaggctccattgtgagcatgtctgatttagagtgcttggtaagaaataatacccgtgacaggggg